(19) United States
(12) Patent Application Publication
(10) Pub. No.: US 2010/0291683 A1
(43) Pub. Date: Nov. 18, 2010

(54) Modified Antigen Presenting Cells and Methods of Use

(76) Inventors: Lung-Ji Chang, Gainesville, FL (US); Bei Wang, Gainesville, FL (US); Shuhong Han, Gainesville, FL (US)

Correspondence Address: Beusse Wolter Sanks Mora & Maire, 390 N. ORANGE AVENUE, SUITE 2500, ORLANDO, FL, 32801, US

(21) Appl. No.: 12/530,340

(22) PCT Filed: Mar. 27, 2008
(86) PCT No.: PCT/US08/58445

§ 371 Date: May 12, 2010

Related U.S. Application Data
(60) Provisional application No. 60/908,258, filed on Mar. 27, 2007.
(60) Provisional application No. 61/025,121, filed on Jan. 31, 2008.

(51) Int. Cl. C12N 15/63; C12N 5/10

(52) U.S. Cl. 435/455; 435/325

(57) ABSTRACT

The invention provides compositions comprising and methods of using antigen presenting cells into which has been introduced at least a first nucleotide sequence that encodes calnexin, wherein expression of calnexin in the antigen presenting cell increases the antigen presenting cell's ability to activate a T cell response.

### FIELD OF THE INVENTION

The invention relates to the fields of molecular biology, gene therapy, immunology, and virology. More particularly, the invention relates to compositions and methods for modulating a cancer cell-specific immune response using antigen presenting cells into which a nucleic acid encoding calnexin has been introduced.

### BACKGROUND OF THE INVENTION

The standard chemotherapy and radiation therapy used in cancer treatment only offer limited control of the disease. For example, multiple myeloma (MM), an incurable B-cell neoplasm, accounts for approximately 1% of all cancers but it is the second most common hematologic malignancy after lymphoma. In MM patients, the malignant plasma cells accumulate in the bone marrow leading to lytic bone lesions, anemia and excessive amounts of monoclonal immunoglobulins (Igs) such as IgG, IgA or free light chains. Conventional chemotherapy and autologous transplant used in MM treatment (Hideshima et al., Immunol Rev. 194:164-176, 2003; Denz et al., Eur J Cancer 42:1591-1600, 2006) have had limited success. Another strategy for treating MM is allogeneic stern cell transplant (allo-SCT). However, allo-SCT is associated with high treatment-related mortality and limited to individuals with HLA-matched donors. Furthermore, myeloma cells may elude destruction in a large number of patients undergoing this treatment, and consequently, most patients eventually die from recurrent disease.

Immunotherapy is considered an alternative means to treating malignancies and has been the focus of multiple studies (Raje et al., Br J. Haematol. 125:343-352, 2004; Bogen et al., Haematologica 91:941-948, 2006; and Houet and Veelken, Eur J Cancer 42:1653-1660, 2006). Dendritic cells (DCs) are a key player in establishing an effective anti-cancer immunity. In addition to stimulating T cell responses, DCs also activate innate immunity such as NK and NKT cells. The DC immunization approach for cancer therapy has gained only limited success because many patients have established strong immune tolerance toward their cancer cells. Thus, a successful and effective immunotherapy for treating cancer is needed.

### SUMMARY

The invention relates to the discovery that DCs expressing supraphysiological levels of calnexin (CNX) via a lentiviral gene transfer system stimulated expansion of high-avidity CTLs with increased central memory phenotype. Compared with unmodified DCs, CNX-DCs expressed increased amounts of adhesion and antigen presentation molecules with the ability to prime T cells to exhibit increased functional avidity and upregulation of CCR7 and costimulatory TNF receptor superfamily molecules. In a Balb/c mouse tumor model, significant tumor regression was observed when CNX-DCs were used to present tumor antigens. The invention also relates to the discovery that DCs from cancer patients suppress rather than induce a cancer cell-specific immune response. Cancer cell lysates and cancer-specific antigen suppressed an anti-myeloma immune response, specifically inducing expansion of peripheral CD4⁺CD25^{high}FoxP3^{high} T regulatory (Treg) cells. Supraphysiological expression of calnexin, a chaperone molecule essential to glycoprotein processing in the endoplasmic reticulum, in DCs of cancer patients using lentiviral delivery of the calnexin gene overcame the immune suppression and enhanced cancer cell-specific CD4 and CD8 T cell responses. T cells primed with dendritic cells expressing supraphysiological levels of calnexin exhibited increased functional avidity maturation and CCR7 expression. These T cells also exhibited an upregulation of costimulatory molecules belonging to the TNF receptor superfamily. This increased T cell immunity was translated into therapeutic efficacy in a murine tumor model and resulted in an enhanced anti-cancer immune response in human cancer patients' cells ex vivo. Described herein are modified antigen presenting cells expressing higher than normal levels of calnexin as immune modulatory cells which induce an effective anti-cancer immunity.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of virology terms can be found in Granoff and Webster, Encyclopedia of Virology, 2nd edition, Academic Press: San Diego, Calif., 1999; and Tidona and Darai, The Springer Index of Viruses, 1st edition, Springer-Verlag: New York, 2002. Commonly understood definitions of microbiology can be found in Singleton and Sainsbury, Dictionary of Microbiology and Molecular Biology, 3rd edition, John Wiley & Sons: New York, 2002.

As used herein, the phrase “nucleic acid” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced by polymerase chain reaction (PCR), nucleic acids formed by restriction enzyme treatment of genomic nucleic acids, recombinant nucleic acids, and chemically synthesized nucleic acid molecules.

As used herein, the term “vector” refers to an entity capable of transporting a nucleic acid and/or a virus particle, e.g., a plasmid or a viral vector.

By the term “supraphysiological” is meant expression of a protein at levels higher than normal cellular physiological levels, for example, 15,000 molecules rather than 10,000 molecules per cell.

As used herein, the phrase “cancer cell-specific activating activity of a DC” refers to an antigen presenting cell's ability to induce a specific anti-cancer immunity through modified antigen presenting cells, e.g., a modified dendritic cell capable of inducing a cancer antigen-specific cytotoxic T cell response, or a modified dendritic cell capable of inducing an anti-virus specific cytotoxic T cell response.

Although methods and compositions similar or equivalent to those described herein can be used in the practice or testing of the present invention, suitable methods and compositions are described below. All publications, patent applications, patents and other references mentioned herein are incorporated by reference in their entirety. In the case of conflict, the present specification, including definitions will control.

### BRIEF DESCRIPTION OF THE DRAWING

FIG. 1A is a diagram of MM cell lysate preparation and DC and T cell co-culture.

FIG. 1B shows the results from a flow cytometry analysis of purified CD38⁺CD138⁺CD56⁺CD45⁻ MM cancer cells. The cancer cells were isolated from bone marrow of MM patients and purified with Ab-labeled magnetic beads. The purified MM cancer cells were analyzed by surface staining for CD38, CD138, CD56 and CD45, before and after magnetic bead selection. The numbers denote percentages of the specific cell population. The result is a representative of five cancer patients.

FIG. 1C shows the results from a comparison of DC surface phenotype between healthy donors (HD) and MM cancer patients (MM). Blood monocytes were isolated and cultured with GM-CSF (50 ng/ml and IL-4 (25 ng/ml) for 5 days. DC maturation was induced with TNFα (20 U/ml) and LPS (1 ug/ml) for 48 hours. The surface phenotype was analyzed with fluoresence-conjugated antibodies against CD11c, CD1a, CD83, CD80, HLA-I, CD86, CD40 and HLA-DR. The gray areas represent isotype controls.

FIG. 2A is a series of graphs showing intracellular cytokine staining (ICCS) of DC-activated T cells. On day 14, the cells were re-stimulated with PMA and ionomycin and the number of CD4⁺ or CD8⁺T cells secreting TNFα and IFNγ was determined by ICCS.

FIG. 2B is a series of graphs and plots showing expansion of Treg cells by cancer cell lysates. On day 13, the frequency of CD4⁺CD25^{high}FoxP3^{high}Treg cells was analyzed with fluorescence conjugated antibodies against CD4, CD25 and FoxP3. The CD4⁺CD25^{high} T cells were gated and the expression of FoxP3 was analyzed. The result represents triplicate assays from four cancer patients' specimens (*, p<0.05 and **, p<0.001).

FIG. 3A is a diagram of the strategy of cloning MM cancer cell Id-Ig cDNA.

FIG. 3B is a schematic illustrating PCR identification of MM cancer-specific kappa chain cDNA.

FIG. 3C is the amino acid and nucleotide sequence of the MM cancer cell kappa chain cDNA. The CDR3 region of the MM kappa gene is underlined.

FIG. 4A is a diagram showing the strategy of verification of the MM cancer cell Id-Ig gene.

FIG. 4B is a photograph of an electrophoretic gel showing PCR verification of the MM Id-Ig gene. Lane 1, DNA from a specific cancer (MM3) patient's bone marrow cells; lane 2, DNA from MM3 patient's bone marrow-derived stromal cells; lane 3, DNA from the control MM patient's (MM4) bone marrow-derived stromal cells.

FIG. 4C is a diagram of lentiviral expression of the cancer antigen Kappa-Flag fusion protein and a photograph of a Western blot showing expression of the Kappa-Flag fusion protein. The illustrated lentiviral self-inactivating (SIN) vector construct (pTYF-EF-k-Flag) was used to transduce 293T cells. The expression of the Kappa-Flag fusion protein was confirmed by Western analysis using an anti-Flag antibody.

FIG. 5A is a series of graphs showing the results of an analysis of CD4 and CD8 T cell response by ICCS for the Ag-specific expression of TNFα and IFNγ.

FIG. 5B is a series of plots and graphs showing intracellular staining of FoxP3 for the detection of CD4+CD25high Treg cells. The experiments were repeated three times and a representative result is shown (*, p<0.05 and **, p<0.001).

FIG. 6A is a photograph of a Western blot showing the detection of calnexin expression after LV-CNX gene transfer. LV-CNX was used to infect CEM-NKR cells, a calnexin-deficient cell line. After 96 hr, the expression of calnexin was verified by Western analysis. The calnexin and the control α-tubulin were detected using specific monoclonal antibodies.

FIG. 6B is a series of graphs showing that LV-CNX transduced DCs enhance cancer cell-specific T cell immunity. Immature DCs were infected with LV-LacZ or LV-CNX (DC-LV-LacZ and DC-LV-CNX), and pulsed with MM lysates for 4 hr (DC-LV-LacZ/MM and DC-LV-CNX/MM). After co-culture with autologous non-adherent PBMC for 14 days, the T cells were stimulated with PMA and inomycin and the CD4 and CD8 T cells secreting TNFα and IFNγ were evaluated. The assay result represents triplicates of four separate MM cancer patients' specimens (*, p<0.05 and **, p<0.001).

FIG. 7A is a series of photographs showing that CNX-DCs promote T cell proliferation. The cocultured cells were photographed in a 96-well culture plate under an inverted microscope (5×15). The proliferation of T cells was detected by analyzing CFSE-labeled peripheral blood mononuclear cells (PBMCs) after three days in coculture. The mean fluorescence index is shown in the FACS graph.

FIG. 7B is a pair of graphs showing that LV-CNX transduced DCs up-regulate effector T cell functions. On day 14, the T cells were re-stimulated with the same antigen-treated mature DCs. The number of CD4⁺ and CD8⁺ T cells secreting TNFα and/or IFNγ was analyzed by ICCS. The response to DC-LV-LacZ and to DC/TT is considered primary and memory response, respectively. The results represent one of three assays (*, p<0.05 and **, p<0.001).

FIG. 7C is a pair of graphs showing that LV-CNX transduced DCs induce increased CTL activity targeting a cancer cell-specific antigen, MM Id-Ig. On day 14 after coculture, the T cells were re-stimulated for 5 days. The T cells were harvested as effecter cells and incubated with target cells including primary MM cancer cells isolated with magnetic beads, or autologous stromal cells infected with LV-Kappa. The control cells are autologous EBV transformed B cells and stromal cells infected with LV-LacZ. The CTL killing effect was measured with FATAL assay. The assay was repeated three times and a representative result is shown (*, p<0.05).

FIG. 8 is a series of graphs and plots showing results that suggest that cancer patients' DCs over-expressing CNX do not suppress Treg cell expansion induced by cancer cell lysates

FIG. 9 is a series of graphs and plots showing results that suggest that cancer patients' DCs over-expressing CNX do not suppress Treg cell expansion induced by cancer cell-specific antigen MM Id-Ig

FIG. 10A is a schematic diagram of DC modifications for T cell stimulation. Day 5 immature DCs were transduced with different LVs and induced maturation in day 6 by LPS and TNF-α. At day 7, mature DCs were directly used to stimulate T cell or loaded with GLC peptide as indicated.

FIG. 10B shows an immunoblot (top) and flow cytometry (lower) analysis of the expression of CNX in CEM-NKR cells and DCs transduced with mock or LV-CNX. Total α-tubulin serves as the internal control in immunoblot. The black line represents isotype control; blue, mock; red, LV-CNX.

FIG. 10C is a series of graphs showing allogeneic MLR. Allogeneic T cells (2×10⁵) were co-cultured with mock, LV-Ctrl or LV-CNX transduced DCs at 20:1 ratio for 6 hours. CD8 and CD4 T cell activation was quantified by IL-2, TNF-α, IFN-γ intracellular cytokine staining Data are mean±s.d of four experiments.

FIG. 10D,E are plots and graphs showing representative results of flow cytometry and frequency, respectively, of HLA-A2 BMLF-1 GLC pentamer-positive antivirus T cells. T cells were primed by transduced DCs as described in FIG. 10A. Twelve days after stimulation T cells were stained with HLA-A2 GLC pentamer combined with anti-CD8 antibody and analyzed by flow cytometry. Numbers shown are percentage of CD8⁺ T cells that were pentamer-positive. Results are representative of four experiments. *P<0.05; ***P<0.005.

FIG. 11A is a pair of graphs showing results from a proliferation assay of primed T cells. Day 12 primed T cells were labeled with CFSE and restimulated with peptide-loaded BLCL cells at different ratios. At day 6, cells were harvested, labeled with GLC pentamer, anti-CD8 antibody and analyzed by flow cytometry. GLC pentamer positive antivirus T cells were gated and further analyzed for proliferation index.

FIG. 11B is a series of plots showing quantification of IFN-γ and TNF-α production of CD8⁺ T cells. Day 12 primed T cells were restimulated with control of GLC peptide and intracellular IFN-γ and TNF-α production were quantified by intracellular cytokine staining Data are percentage of cytokine producing CD8⁺ T cells in total T cells.

FIG. 11C is a pair of graphs showing representative flow cytometry and frequency of IFN-γ and TNF-α producing cells in gated GLC pentamer positive antivirus CD8+ T cells after restimulation with control or GLC peptide. The percents of cell population are indicated in the FACS quadrants.

FIG. 11D is a graph showing results from a CTL assay. Primed T cells were restimulated for 5 days and harvested as effector cells. Target cells are autologous BLCL loaded with control or GLC peptide. FATAL assays, as described in Methods, were performed to compare the cytolytic function of T cells. Data are representatives of four experiments. *P<0.05; **P<0.01.

FIG. 12A shows the immunophenotype of DC/LV-CNX. The d5 DCs were transduced with LV-Ctrl, LV-CNX or no vector control (mock). Twenty-four hours after infection DCs were treated with TNF-α/LPS for 48 hours and subsequently analyzed for their phenotype by direct immunofluorescence staining. One representative experiment of six experiments is shown. Gray lines, isotype control staining

FIG. 12B is a series of plots showing that DC/LV-CNX upregulate HLA class I surface expression and flow cytometry of HLA class I expression in DCs. Numbers indicate the percent of gated cells (HLA-I+ or HLA++). One representative experiment of six experiments is shown.

FIG. 12C-D show results from a quantitative analysis of HLA class I expression. (c) Mean fluorescence index (MFI) of HLA class I. (d) Percent of HLA-I++ DCs.

FIG. 13A,B show a decreased activation threshold of CD8⁺ T cells primed by CNX engineered DCs. Intracellular IFN-γ staining was performed after 6 h stimulation with indicated peptide concentration. Representative flow cytometry (A) and quantification data (B) were shown.

FIG. 13C shows results from a functional avidity analysis. IFN-γ production by GLC virus-specific T cells was quantified 6 h after restimulation with the indicated doses of peptide. The result expressed as a percentage of the maximum response attained with the 5×10⁻⁶ M peptide concentrations. The dotted lines represent the peptide concentration required to obtain 50% of maximum cytokine production. Results are representatives of four experiments.

FIG. 14A shows surface phenotypes of Ag-specific T cells. Primed T cells were restimulated for 7 days and stained with GLC-pentamer and antibodies against CCR7, CD62L, CD28 and CD69. GLC pentamer-positive antivirus T cells were gated and analyzed for expression of surface differentiation markers. The percent of cell population are indicated in the FACS quadrants.

FIG. 14B shows quantification of CCR7⁺ cells in pentamer-positive T cells. Data are representative of four experiments.

FIG. 15A is schematic diagrams of PCR array analysis. Pentamer-positive T cells were purified by magnetic beads sorting. Purity of the sorted cells is indicated in the FACS quadrants. The purified T cells were further stimulated with specific peptide for five rounds and total RNA were harvested seven days after last stimulation for PCR array analysis.

FIG. 15B shows a selected summary of PCR array analysis of purified GLC pentamer positive antivirus T cells primed by LV-GFP/LV-BMLF-DCs (T_{Ctrl}) or LV-CNX/LV-BMLF-DCs (T_{CNX}).

FIG. 16A illustrates improving tumor vaccine efficacy in vivo. Mice were first challenged with 1×10⁵ CT26/E6E7 tumor cells subcutaneously. One week later, mice were given 2.5×10⁵ DCs transduced with LV-nLacZ, LV-opiE6E7, or LV-CNX/LV-opiE6E7, weekly for 3 weeks. The tumor size was measured over time using caliper and the mean tumor volume (in mm³) was determined. Error bars depict standard deviation (SD), n=5.

FIG. 16B shows enhancement of cancer-specific CD8 T cell responses in vivo. Mice splenocytes were harvested 25 days after tumor cell injection, pooled and restimulated with colon cancer cell CT26 or colon cancer cells expressing human papilloma virus antigen E6 and E7, CT26-E6E7. Intracellular cytokine staining for IFN-γ, and TNF-α were performed and quantified by FACS. The experiments were repeated twice. (c) Frequency of HLA-A2 HPA16 E7 tetramer-positive T cells. T cells from late stage cervical carcinoma patient were stimulated with DCs transduced with mock, LV-Ctrl, LV-CNX, LV-opiE6E7, or LV-opiE6E7/LV-CNX and harvested at day 14 for tetramer staining (d) FATAL assay. Primed T cells were restimulated with LV-opiE6E7-DCs for 5 days and used as effector cells. Target cells were LV-opeE6E7-DCs. Data are representatives of three experiments. *P<0.05.

FIG. 17 is a table showing surface phenotypes of DC-CNX.

FIG. 18 is a comparison of DC surface phenotype between healthy donors (HD) and MM patients (MM). The percentages of positive cells against isotype Ab controls are summarized in the bottom.

FIG. 19 shows that DCs transduced with LV-MM Id-Ig suppress CD4 and CD8 T cell response and up-regulate Treg response. (A) Analysis of Ag-specific effector cell response. The immature DCs from MM patients (MM) and healthy donors (HD) were transduced with LV-LacZ (DC-LV-LacZ), LV-Kappa (DC-LV-Kappa) or pulsed with TT (DC/TT), and co-cultured with autologous non-adherent PBMCs for 14 days. For specific response, the T cells were re-stimulated with the same antigen-treated DCs. CD4 and CD8 T cell secreting TNF-α and IFN-γ are analyzed by ICC\tilde{S}'. (B). Dose-dependent suppression of PBMC proliferation by CD4⁺CD25⁺ T cells. CFSE labeled PBMCs were activated with PHA and incubated with CD4⁺CD25⁺ or CD4⁺CD25⁻T cells at increasing ratios as indicated. After 3 days, the proliferation of PBMCs was measured based on CFSE intensity. Representative CFSE profile from one MM patient is shown (n=3, plus three healthy donors); M1 represents the percentage of dividing cells. (C). Bar graph analysis of suppression of PBMC proliferation (one of three assays).

FIG. 20 is a pair of graphs showing that LV-CNX transduced DCs induce increased CTL activity targeting MM cells. The % specific lysis=% lysis of target cells with specific antigen−% lysis of control target cells. Representative of three assays is shown (*, p<0.05).

FIG. 21 is a series of graphs showing that calnexin alone does not non-specifically up-regulate effector cell functions, but supraphysiological expression of CNX in DCs promotes T cell response against known tumor antigens (HPV E6 and E7).

### DETAILED DESCRIPTION

The invention provides methods and compositions for modulating an antigen presenting cell's (e.g., DC) ability to activate a cancer cell-specific or virus-specific T cell response. The activation of specific anti-cancer immunity using genetically modified DCs from cancer or virus-infected patients may be used to overcome immune tolerance against cancers such as multiple myeloma or virus infection such as human hepatitis C virus.

The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### Proteins Capable of Modulating a DC's Ability to Activate a Cancer Cell-Specific or Virus-Specific Immune Response

The invention relates to the use of antigen presenting cells (e.g., DCs) into which have been introduced a nucleotide sequence encoding calnexin. A nucleic acid encoding calnexin can be introduced into antigen presenting cells (e.g., DCs) by any suitable vector or construct. In one embodiment, the purified nucleic acid is included within a lentivirus. In such an embodiment, a construct including a first nucleotide sequence from a lentivirus and a second nucleotide sequence that encodes calnexin. In this embodiment, calnexin is capable of modulating the antigen presenting cells' (e.g., DCs) ability to activate a cancer cell-specific (e.g., multiple myeloma cell-specific). Such a construct can further include defensin; in some embodiments, nucleotide sequences encoding both calnexin and defensin are introduced into the DCs. The first and second nucleotide sequences are typically within a lentiviral vector.

Immune tolerance to cancer-associated antigens may arise from immune ignorance or the deletion or functional inactivation (anergy) of cancer-specific T cells (Mapara and Sykes, J Clin Oncol 22:1136-1151, 2004; Modino et al., Proc Natl Acad Sci USA 93:2245-2252, 1996). The results described in the Examples section indicate that cancer cell lysates or cancer cell-specific Id-Ig induce a Treg cell response instead of an anti-MM response. A growing body of evidence now supports the induction and expansion of regulatory T cells as a mechanism responsible for the lack of a clinically-sufficient anti-cancer immune response (Beyer and Schultze, Blood 108:804-811, 2006). Among the CD4⁺ Treg cells, T regulatory type 1 (Tr1) cells have been shown to down-modulate immune responses through the action of immunosuppressive cytokines IL-10 and TGF-β. Tr1 cells maintain peripheral tolerance, control autoimmunity, and prevent allograft rejection and graft versus host disease (GvHD). Control of Treg cell expansion may facilitate the development of anti-cancer immunity.

As described in the Examples section, cancer cell lysate-pulsed DCs effectively up-regulated Treg cells. The expansion of Treg cells is further demonstrated with DCs expressing specific cancer cell-specific Id-Ig. These expanded Treg cells expressed increased amount of intracellular FoxP3 with a CD4⁺CD25^{high} phenotype. Thus, the results revealed that the overall immune effector functions are reduced when patients' DCs are exposed to the cancer antigens, indicating that a strong immune tolerance has developed in these cancer patients.

Cancer patients gradually develop tolerance to their cancer cells with increased Treg cell activities. This tilted balance of cancer immunity may be altered by engineering DCs to express supraphysiological levels of calnexin. In addition, the function of CD4⁺CD25^{high}FoxP3⁺ Treg cells could be effectively inundated by the modified anti-cancer immunity. The results also show that the cancer patients' immune tolerance can be overcome by modifying DCs to express calnexin, an accessory protein that enhances antigen processing and promotes DC and T cell interaction. Calnexin is a chaperone facilitating glycoprotein processing in antigen presenting cells. Calnexin plays a key role in both MHC-I and MHC-II antigen processing pathways and may also be involved in CD1d lipid antigen presentation (Bouvier, Mol Immunol 39:697-706, 2003; Kang and Cresswell, J Biol Chem 277:44838-44844, 2002). Calnexin may also enhance cross-presentation of exogenous antigens through the MHC-I pathway (Wan et al., Eur J Immunol 35:2041-2050, 2005). Without modifications, cancer patients' DCs presenting cancer antigens do not induce an effective T helper or CTL response against cancer cells. After modification with LV-calnexin, the cancer patients' DCs effectively boosted cytokine production in both CD4 and CD8 T cells coupled with increased cancer cell killing activity. These findings indicate that the tolerogenic DCs in cancer patients may be engineered into reactive DCs to promote an anti-cancer immunity. Because effective anti-cancer immunity requires both CD4 and CD8 T cells and the results described in the Examples section show that calnexin-modified DCs enhance both CD4 and CD8 T cell activities, DCs modified with LV-calnexin may be used to activate a strong anti-cancer immunity with potential clinical benefit.

As described in the Examples below, T cells primed with dendritic cells expressing supraphysiological levels of calnexin exhibited increased functional avidity maturation and CCR7 expression. These T cells also exhibited an upregulation of costimulatory molecules belonging to the TNF receptor superfamily. This increased T cell immunity was translated into therapeutic efficacy in a murine tumor model and resulted in an enhanced anti-cancer immune response in human cancer patients' cells ex vivo.

The results described herein support the application of calnexin-based immunotherapy in multiple myeloma and potentially other malignancies as well. Together with specific depletion of cancer-specific Treg populations, an engineered cancer therapeutic DC vaccine may tip the balance in favor of cancer eradication.

### Nucleic Acids and LVs

The invention provides nucleotide sequences that encode calnexin, and nucleotide sequences from lentivirus. In one example, a nucleic acid (e.g., a vector) includes a first nucleotide sequence derived from a lentivirus and a second nucleotide sequence that encodes calnexin which is capable of modulating the DCs' ability to activate a cancer cell-specific (e.g., multiple myeloma cell-specific) T cell response. Nucleic acids encoding calnexin are known, e.g., GenBank accession number P27824. A vector as described herein typically takes the form of a LV. A number of different types of LVs are known including those based on naturally occurring lentiviruses such as HIV-1, HIV-2, simian immunodeficiency virus (SIV), feline immunodeficiency virus (FIV), bovine immunodeficiency virus (BIV) and others. See U.S. Pat. No. 6,207,455. Although the invention is described using HIV-1 based vectors, other vectors derived from other lentiviruses might also be used by adapting the information described herein. HIV-1 based vectors provide many advantages for gene and cell-based therapy applications, including efficient transduction of different types of cells, high level of transgene expression and long term stable proviral integration.

The LVs of the invention might be pseudotyped, e.g., to overcome restricted host cell tropism. For example, LVs pseudotyped with vesicular stomatitis virus G (VSV-G) viral envelopes might be used. To enhance safety, a SIN LV might also be used. For example, SIN LVs can be made by inactivating the 3′ U3 promoter and deleting of all the 3′ U3 sequence except the 5′ integration attachment site which is important for integration into the host chromosome. Particularly useful constructs for designing vectors of the invention are the SIN pTYF vectors (see, Chang et al., Gene Ther. 6:715-728, 1999; Zaiss et al., J. Virol. 76:7209-7219, 2002; and Chang L-J, Zaiss A-K, Self inactivating lentiviral vectors in combination with a sensitive Cre/loxP reporter system. In: Walker J, ed., Methods in Molecular Medicine Humana Press Inc., 367-382, 2001).

Construction of recombinant LVs and virions is discussed in Buchschacher et al., Blood 95:2499-2504, 2000; Chang et al., Gene Therapy 6:715-728, 1999; Emery et al., PNAS 97:9150-9155, 2000; Naldini et al., Science 272:263-267, 1996; Paillard et al., 9:767-768, 1998; Sharma et al., PNAS 93:11842-11847, 1996; Reiser et al., PNAS 93:15266-15271, 1996; and Chinnasamy et al., Blood 96:1309-1316, 2000. SIN vector design is described in Miyoshi et al., J. Virol. 72:8150-8157, 1998; Zufferey et al., J. Virol. 72: 9873-9880, 1998; Iwakuma et al., Virology 261:120-132, 1999; Mangeot et al., J. Virol. 74:8307-8315, 2000; and Schnell et al., Hum. Gene Ther. 11:439-447, 2000.

Any additional nucleotide sequence elements which facilitate expression of calnexin in DCs and cloning of a vector encoding calnexin can be used in the compositions and methods described herein. The presence of enhancers upstream of the promoter or terminators downstream of the coding region, for example, can facilitate expression.

### Antigen Presenting Cells

The invention provides an antigen presenting cell (e.g., DC) into which has been introduced a purified nucleic acid encoding calnexin. Antigen presenting cells (e.g., DCs) that may be used include mammalian antigen presenting cells such as those from mice, rats, guinea pigs, non-human primates (e.g., chimpanzees and other apes and monkey species), cattle, sheep, pigs, goats, horses, dogs, cats, and humans. The antigen presenting cells may be those within a mammalian subject (i.e., in vivo), or those within an in vitro culture (e.g., those cultured in vitro for ex vivo delivery to a subject).

Antigen presenting cells such as DCs can be obtained from any suitable source, including the skin, spleen, bone marrow, or other lymphoid organs, lymph nodes, or blood. Generally, DCs are obtained from blood or bone marrow for use in the compositions and methods described herein. Typically, DCs are generated from bone marrow and PBMCs after stimulation with exogenous granulocyte-macrophage colony stimulating factor (GM-CSF) and interleukin-4 Methods for obtaining DCs from bone marrow cells and culturing DCs are described in Inaba et al., J. Exp. Med. 176:1693-1702, 1992; and Bai et al., Int. J. Oncol. 20:247-253, 2002. Methods for culturing DCs from hematopoietic progenitor cells (Mollah et al., J. Invest. Dermatol. 120:256-265, 2003) and monocytes (Nouri-Shirazi and Guinet Transplantation 74:1035-1044, 2002) are also known in the art. An example of a large-scale monocyte-enrichment procedure for generating DCs is described in Pullarkat et al. (J. Immunol. Methods 267:173-183, 2002). DCs may be isolated from a heterogeneous cell sample using DC-specific markers in a fluorescence-activated cell sorting (FACS) analysis (Thomas and Lipsky J. Immunol. 153:4016-4028, 1994; Canque et al., Blood 88:4215-4228, 1996; Wang et al., Blood 95:2337-2345, 2000). Immature DC are characterized by low level expression of costimulatory molecules, CD80/86, CD40; poor ability to induce T cell activation; inability to produce IL-12p70; and the potential to induce regulatory or anergic T cells. In comparison, mature DC produce IL-12p70 and express high levels of MHC class II antigens, CD80/86, and CD40, IL-12p70 production. A population of cells containing DCs as well as isolated DCs may be cultured using any suitable in vitro culturing method that allows growth and proliferation of the DCs.

### Introducing Nucleic Acids Encoding Calnexin into DCs

Described herein are compositions and methods involving the introduction of a nucleic acid encoding calnexin into antigen presenting cells (e.g., DCs) such that the antigen presenting cells express calnexin at supraphysiological levels. The compositions as described herein can be introduced into antigen presenting cells (e.g., DCs) by any suitable technique. Various techniques using viral vectors for the introduction of a nucleic acid encoding calnexin into cells are provided for according to the invention. Although lentiviral vectors were used in the Examples described herein, additional viral vectors can be used to transduce DCs or DC progenitor cells with a nucleic acid encoding calnexin, resulting in supraphysiological expression of calnexin in the DCs. Examples of additional viral vectors include Adenoviruses (Amalfitano A. and Parks R. J., Curr Gene Ther 2:111-133, 2002; W. C. Russell, Journal of General Virology 81:2573-2604, 2000, and Bramson et al., Curr. Opin. Biotechnol. 6:590-595, 1995), Adeno-Associated Virus (AAV) vectors (Tal, J., J. Biomed. Sci. 7:279-291, 2000 and Monahan and Samulski, Gene Therapy 7:24-30, 2000), Herpes Simplex Virus (HSV) vectors (Cuchet et al., Expert Opin Biol Ther 7:975-995, 2007), Murine Leukemia Virus-based vectors and other retrovirus vectors (Dalba et al., Curr Gene Ther 5:655-667, 2005), and Alphaviruses, including Semliki Forest Virus and Sindbis Virus (Lundstrom, K., Intervirology 43:247-257, 2000 and Perri et al., Journal of Virology 74:9802-9807, 2000). Viral vector methods and protocols are reviewed in Kay et al. Nature Medicine 7:33-40, 200; Mandel et al., Exp Neurol. 209:58-71, 2008; Lawson, C., Methods Mol Biol 333:175-200, 2006; and Wong et al., Human Gene Ther 17:1-9, 2006.

To combine advantageous properties of two viral vector systems, hybrid viral vectors may be used to deliver a canexin gene to DCs. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and Adenoviral ITRs may be used to transduce cells. In another variation, an AAV vector may be placed into a “gutless”, “helper-dependent” or “high-capacity” Adenoviral vector. Adenovirus/AAV hybrid vectors are discussed in Lieber et al., J. Virol. 73:9314-9324, 1999. Retroviral/Adenovirus hybrid vectors are discussed in Zheng et al., Nature Biotechnol. 18:176-186, 2000.

Any suitable non-viral method for introducing a nucleic acid encoding calnexin into DCs can be used in compositions and methods described herein. For a review of non-viral methods, see Nishikawa and Huang, Human Gene Ther. 12:861-870, 2001; Nishikawa et al., Cancer Sci. 2008 Feb. 19 [Epub ahead of print]; Wagstaff K M and Jans D A, Biochem J. 406:185-2002, 2007; and Kodama et al., Curr Med Chem 13:2155-2161, 2006. Methods involving physical techniques for the introduction of a nucleic acid encoding calnexin into DCs can be adapted for use in the present invention. The particle bombardment method of gene transfer involves an Accell device (gene gun) to accelerate DNA-coated microscopic gold particles into target tissue, including the liver. Particle bombardment methods are described in Yang et al., Mol. Med. Today 2:476-481 1996 and Davidson et al., Rev. Wound Repair Regen. 6:452-459, 2000. Cell electropermeabilization (also termed cell electroporation) may be employed for gene delivery into DCs. This technique is discussed in Preat, V., Ann. Pharm. Fr. 59:239-244 2001; and Isaka Y. and Imai E., Expert Opin Drug Deliv. 4:561-571, 2007, and involves the application of pulsed electric fields to cells to enhance cell permeability, resulting in exogenous polynucleotide transit across the cytoplasmic membrane.

Synthetic gene transfer molecules according to the invention can be designed to form multimolecular aggregates with plasmid DNA (encoding calnexin) and to bind the resulting particles to the target cell (i.e., DC) surface in such a way as to trigger endocytosis and endosomal membrane disruption. Polymeric DNA-binding cations (including polylysine, protamine, and cationized albumin) can trigger receptor-mediated endocytosis into DCs. Methods involving polymeric DNA-binding cations are reviewed in Garnett, M. C., Crit. Rev. Ther. Drug Carrier Syst. 16:147-207, 1999, and Eliyahu et al., Molecules 10:34-64, 2005. Methods involving cationic lipid formulations are reviewed in Feigner et al., Ann. N.Y. Acad. Sci. 772:126-139, 1995 and Lasic and Templeton, Adv. Drug Delivery Rev. 20:221-266, 1996. Suitable methods can also include use of cationic liposomes as agents for introducing DNA or protein into cells. For therapeutic gene delivery, DNA may also be coupled to an amphipathic cationic peptide (Fominaya et al., J. Gene Med. 2:455-464, 2000).

Methods that involve both viral and non-viral based components may be used according to the invention. An Epstein Barr Virus (EBV) based plasmid for therapeutic gene delivery is described in Cui et al., Gene Therapy 8:1508-1513, 2001. A method involving a DNA/ligand/polycationic adjunct coupled to an Adenovirus is described in Curiel, D. T., Nat. Immun. 13:141-164, 1994.

### Increasing a Cancer Cell-Specific Activating Activity of a DC in a Subject

Compositions and methods for increasing a cancer cell-specific activating activity of a DC in a subject may be used in a variety of DC-based immunotherapy strategies for treating different cancers. Mature DC are the key antigen presenting cell population which efficiently mediates antigen transport to organized lymphoid tissues for the initiation of T cell responses (e.g., induction of cytotoxic T lymphoctyes). The normal function of DCs is to present antigens to T cells, which then specifically recognize and ultimately eliminate the antigen source. DCs are used as both therapeutic and prophylactic vaccines for cancers and infectious diseases. Such vaccines are designed to elicit a strong cellular immune response. DC biology, gene transfer into DC, and DC immunotherapy are reviewed in Lundqvist and Pisa, Med. Oncol. 19:197-211, 2002; Herrera and Perez-Oteyza, Rev. Clin. Esp. 202:552-554, 2002; and Onaitis et al., Surg. Oncol. Clin. N. Am. 11:645-660, 2002. The therapeutic role of DCs in cancer immunotherapy is reviewed in Lemoli et al., Haematologica 87:62-66, 2002; A. F. Ochsenbein, Cancer Gene Ther. 9:1043-1055, 2002; Zhang et al., Biother. Radiopharm. 17:601-619, 2002; Di Nicola et al., Cytokines Cell Mol. Ther. 4:265-273, 1998; D. Avigan, Blood Rev. 13:51-64, 1999, and Syme et al., J. Hematother. Stem Cell Res. 10:601-608, 2001.

In a typical method of increasing a cancer cell-specific activating activity of a DC in a subject, the steps include providing a dendritic cell and introducing into the dendritic cell a vector including at least a first nucleotide sequence that encodes calnexin, wherein expression of calnexin in the DC increases the dendritic cell's ability to activate a cancer cell-specific T cell response. In a typical method, calnexin is expressed at supraphysiological levels in the DC (e.g., 1,200 molecules rather than 1,000 molecules per cell). The vector can be any suitable vector, e.g., a lentiviral vector, and can be introduced into the DC using any of the methods described herein for introducing a nucleic acid into a cell.

### EXAMPLES

The present invention is further illustrated by the following specific examples. The examples are provided for illustration only and are not to be construed as limiting the scope or content of the invention in any way.

### Example 1

### Overcoming Immune Tolerance Against Multiple Myeloma with Lentiviral Calnexin-Engineered DCs

### Patients and Donors

Bone marrow and peripheral blood were obtained from patients with newly diagnosed or relapsed/refractory multiple myeloma after informed consent approved by Institutional Review Board (IRB) of University of Florida. Peripheral blood samples from healthy donors (Civitan Blood Center, Gainesville, Fla.) were similarly obtained.

### Generation of Monocyte-Derived DCs and Bone Marrow-Derived Stromal Cells

PBMCs from healthy donors or patients with MM were isolated from buffy coats by gradient density centrifugation in Ficoll-Hypaque (Sigma-Aldrich, St. Louis, Mo.) as previously described (Chen et al., Retrovirology 1:37, 2004). DCs were prepared according to the method of Thurner et al. (Thurner et al., J Immunol Methods, 223:1-15, 1999), with the following modification: on Day 0, five million PBMCs per well were seeded into twelve-well culture plates with serum free AIM-V medium (Invitrogen Corp. Carlsbad, Calif.). The PBMCs were incubated at 37° C. for 2 hr and the non-adherent cells were gently washed off; the remaining adherent monocytic cells were further cultured in AIM-V medium. On day 1, the culture medium was removed with care not to disturb the loosely adherent cells, and 1 ml per well of new AIM-V medium containing 50 ng/ml of recombinant human GM-CSF and 25 ng/ml of IL-4 (Biosource International, Inc. Camarillo, Calif., USA) was added and the cells were cultured at 37° C. under 5% CO₂. On day 3, 1 ml of fresh AIM-V medium containing 100 ng/ml of GM-CSF and 50 ng/ml of IL-4 was added to the culture. On day 5, the non-adherent cells were harvested by gentle pipetting. The purity of immature DCs was determined by fluorescence conjugated anti-CD11c antibody to be about 90%. After washing, the DCs were frozen for later use or used immediately. Immature DCs were induced into maturation with TNFα (20 U/ml) and lipopolysaccharide (LPS) (1 ug/ml). The phenotype of the mature DCs was verified with fluorescence conjugated antibodies against different DC maturation markers.

Bone marrow-derived stromal cells were generated by plating bone marrow mononuclear cells in α-MEM supplemented with penicillin and streptomycin and 20% FBS. The non-attached cells were removed after 4-5 days and the attached cells were propagated as stromal cell culture.

### Isolation of MM Cells and Preparation of MM Cell and PBMC Lysates

Fresh bone marrow aspirates from patients with multiple myeloma were collected in RPMI 1640 supplemented with preservative-free heparin. MM cells were enriched from bone marrow mononuclear cells by negative selection with beads according to the manufacture's instructions (Stem Cell Technologies, Vancouver, BC) or enriched by FACS with the following mixture of antibodies: FITC-labeled anti-human CD38, PE-labeled anti-human CD138, PE-Cy7 labeled anti-CD56 and APC-labeled anti-CD45 mAb (BD Pharmigen, San Diego, Calif.). These cells were lysed by five rounds of freeze-and-thaw between liquid nitrogen and a 37° C. water bath. Cell debris was discarded by centrifugation (20,800 g, 2 min) and the supernatants were stored frozen at −80° C. until use. The lysate from 4×10⁵ MM cells was used to pulse 4×10⁵ immature DCs.

### Generation of Cell Lysate-Pulsed Mature DCs

Monocyte-derived day 5 DCs were incubated with cell lysates at a ratio of 1:1 for 4 h. In some cases, tetanus toxoid (TT, inactivated tetanus toxin) was used to pulse the DCs at a concentration 50 u/ml for 4 hours. Subsequently, DCs were matured with LPS (1 ug/ml) and TNFα (20 U/ml) for 24 h.

### Isolation of and Expression of MM-Specific Idiotype Immunoglobulin (Id-Ig) Gene

The VH and VL fragments of FACS-sorted MM cells were PCR amplified using primers specific for H chain and L chain, followed by cloning and sequencing. The reverse primers of H chain and L chain were complementary to the constant (C) region. The forward primers for H chain and L chain were complementary to the V region of different subfamilies. The sequences of these primers are listed in Table 1.

TABLE 1
PCR primers used for MM Id-Ig gene amplification

V region-specific primers:
(SEQ ID NO: 1)

| | |
|---|---|
| V1a: | 5′ CTC GCA ACT GCC TGC AGG GAC ATC |
| | CAG ATG ACC CAG TCT CC 3′ |

(SEQ ID NO: 2)

| | |
|---|---|
| V2a: | 5′ CTC GCA ACT GCC TGC AGG GAT GTT |
| | GTG ATG ACT CAG TCT CC 3′ |

(SEQ ID NO: 3)

| | |
|---|---|
| V3a: | 5′ CTC GCA ACT GCC TGC AGG GAA ATT |
| | GTG TTG ACG CAG TCT CC 3′ |

(SEQ ID NO: 4)

| | |
|---|---|
| V4a: | 5′ CTC GCA ACT GCC TGC AGG GAC ATC |
| | GTG ATG ACC CAG TCT CC 3′ |

(SEQ ID NO:5 )

| | |
|---|---|
| V5a: | 5′ CTC GCA ACT GCC TGC AGG GAA ACG |
| | ACA CTC ACG CAG TCT CC 3′ |

(SEQ ID NO: 6)

| | |
|---|---|
| V6a: | 5′ CTC GCA ACT GCC TGC AGG GAA ATT |
| | GTG CTG ACT CAG TCT CC 3′ |

Cκ1 (kappa constant region) primer:
(SEQ ID NO: 7)

5′ TCA TCC TCG ACT TGG CCG CCT CGG
CCC TAA CAC TCT CCC CTG TTG AAG CTC
TTT GTG ACG GGC GAT CTC A 3′

K chain specific primers (MM patient):
(SEQ ID NO: 8)

| | |
|---|---|
| 5′ primer: | 5' AAG GAT CCA CCA TGC TCG CAA CTG |
| | CC 3′ |

(SEQ ID NO: 9)

| | |
|---|---|
| 3′ primer: | 5' AAA CTA GTC ACT AAC ACT CTC CCC |
| | TGT TGA AGC 3′ |

MM κ CDR3 specific primer:
(SEQ ID NO: 10)

5′ AGT ATG ATG TTC TCC CAT AC 3′

Kappa-Flag fusion primer:
(SEQ ID NO: 11)

5′ AAA CTA GTC TAC TTG TCG TCA TCG
TCT TTG TAG TCA CAC TCT CCC CTG
TTG 3′:

The amplified cDNA was cloned into the LV vector. To confirm the kappa chain of MM origin, a specific 5′ primer was designed according to the CDR3 sequence of the amplified kappa chain. The 3′ reverse primer is complementary to the C region. These primers were used to amplify cDNAs obtained from BM cells and BM-stromal cells of the MM patient or BM-stromal cells of another MM patient to confirm the origin of the MM Id-Ig gene. The PCR products were analyzed on a 1% agarose gel.

### Lentiviral Vector Preparation and Transduction of DCs

LVs were constructed as described previously (Chang et al., Gene Ther. 6:715-728, 1999; Chang and Zaiss, Methods in Molecular Medicine: Humana Press Inc. 367-382, 2001; and Zaiss et al., J Virol 76:7209-7219, 2002). The self-inactivating pTYF vectors expressing calnexin, MM kappa chain, a Kappa-Flag fusion and nLacZ genes were under the EF1α promoter control. The day 5 immature DCs at 5×10⁵ per well in a 24-well plate containing 200 μl of medium supplemented with GM-CSF (50 ng/ml) and IL-4 (25 ng/ml) were transduced with concentrated LVs at a multiplicity of infection (MOI) of 10-50. The infected cells were incubated at 37° C. for 2 hr with gentle shake every 30 min, and 1 ml of DC medium was added and the culture was incubated with the LVs for an additional 12 hr. DC maturation was induced by adding LPS (1 ug/ml) and TNFα (20 u/ml) and incubated for 24 hr. The mature DCs were harvested with AIM-V medium containing 2 mM EDTA at 37° C. for 20 min, and washed three times with PBS.

### Flow Cytometry Analysis

DCs were incubated with normal mouse serum at room temperature for 30 min and then with specific fluorochrome-conjugated monoclonal antibodies for 30 min. The antibodies used in this study include HLA-ABC (Tu149, mouse IgG2a, FITC-labeled, Caltag Laboratories, Burlingame, Calif.), HLA-DR (TU36, mouse IgG2b, FITC-labeled, Caltag), CD1a (HI49, mouse IgG1k, APC-labeled, Becton Dickinson Pharmigen, San Diego, Calif.), CD80 (L307.4, mouse IgG1k, Cychrome-labeled, BD), CD86 (RMMP-2, Rat IgG2a, FITC-labeled, Caltag), ICAM-1 (15.2, FITC-labeled, Calbiochem), CD11c (Bly-6, mouse IgG1, PE-labeled, BD), CD40 (5C3, mouse IgG1, Cychrome-labeled, BD), and CD83 (HB15e, mouse IgG1, R-PE-labeled, BD). FoxP3 expression was detected using the FITC anti-human FoxP3 Staining Set from eBioscience (San Diego, Calif.). FITC-Rat IgG2a mAbs were used as isotype control. The corresponding isotype control antibodies were included in all staining conditions. After two washes, the cells were resuspended and fixed in 1% paraformaldehyde in PBS and analyzed using a FACSCalibur flow cytometer and the CELLQUEST program (BD).

### DC and Non-Adherent PBMC Coculture

Non-adherent PBMCs were cocultured with autologous mature DCs at a ratio of 20:1 in serum-free AIM-V media for three days. On day 4, IL-7 (10 ng/ml) and IL-2 (12.5 U/ml) were added and new medium containing IL-2 and IL-7 was added every other day for 14 days. On day 13, the T cells were collected for Treg cell analysis using fluorescence conjugated antibodies against CD4, CD25 and FoxP3. On day 14, the T cells were re-stimulated with mature DCs with appropriate antigens. After 18 hr of incubation at 37° C., Golgi-mediated secretion of cytokine was inhibited by the addition of Brefeldin A (1.5 ug/ml), after which the cells were incubated for another 6 hr. For cancer cell lysate-pulsed DCs, the T cells were re-stimulated with PMA (10 ng/ml or 0.0162 μM) and ionomycin (1 μg/ml, Sigma-Aldrich) for 4 hr, with Brefeldin A (1.5 μg/ml) added during the last 2.5 hr of culture. The cells were fixed, permeablized, and stained with FITC-labeled anti-IFNγ-, PE-labeled anti-CD8, PE-Cy7-labeled anti-CD4 and APC-labeled anti-TNF-α mAb (Pharmigen, San Diego, Calif.). The cells were analyzed using a FACSCalibur flow cytometer (BD). T cell cytotoxicity assay (Fluorometric Analysis of T-lymphocyte Antigen-specific Lysis or FATAL assay)

The CTL assay was based on a non-radioactive FATAL assay described by Sheehy et al. (Sheehy et al., J Immunol Methods 249:99-110, 2001), with the following modifications (Wang et al., Vaccine 24:3477-3489, 2006). On day 14 after DC:T cell coculture, the T cells were re-stimulated and 5 days later, harvested as effector cells. The target cells were stromal cell infected with LV-kappa, stromal cells infected with LV-lacZ, MM cells or EBV virus transformed B cell line (BCL). Target cells were first labeled with PKH-26 (Sigma, St. Louis, Mo.). PKH-26 labeled target cells were then labeled with 5-(and -6)-carboxyfluorescein diacetate succinimidyl ester (CFSE) (Molecular Probes, Eugene, Oreg.). The double-labeled target cells were dispensed in duplicate at 5×10⁴ cells/well into 96-well U-bottom plates (Becton Dickinson). Effector cells were added at various E:T ratios and mixed with the target cells. After 5 hr incubation, the cells were harvested and fixed in 1% paraformaldehyde in PBS and analyzed using a FACSCalibur flow cytometer and the CELLQUEST program (BD). PKH-26 positive cells were gated and same cell numbers were acquired for each sample. The percentage of specific cell lysis in the target cell population was determined by the disappearance of the antigen specific targets from the CFSEhi population compared to the control targets in the CFSEhi population. To calculate the percentage of the specific lysis the following equations were used: Percent survival=(mean CFSEhi percent of test well/mean CFSEhi percent of spontaneous release)×100. Percent specific lysis=100−% survival.

### Western Analysis

Cell extracts were prepared in lysis buffer of Cell Signaling Technology, Inc. (Danvers, Mass.) containing proteinase inhibitors (Sigma). The protein samples were separated on sodium dodecyl sulfate 4-12% gradient polyacrylamide gels, electro-blotted to polyvinylidene difluoride membranes (Perkin-Elmer), and exposed to antibodies against Flag or calnexin (Santa Cruz Biotechnology). The signals were detected using antibodies conjugated with horseradish peroxidase and enhanced chemiluminescence (Amersham).

### Statistics

Data were analyzed using GraphPad Prism 4 analysis software and student's t-test. A 2-sided P value of less than 0.05 was considered statistically significant.

### Results

### DC Phenotype Analysis in Healthy Donors and MM Patients

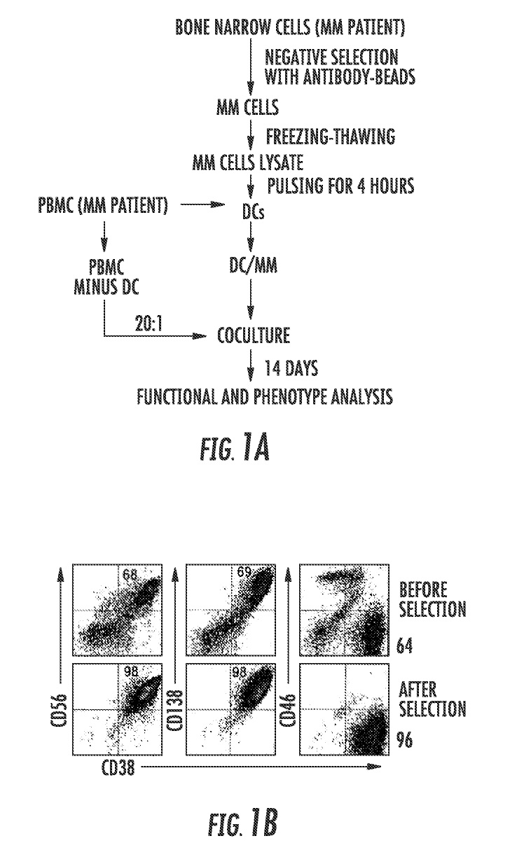

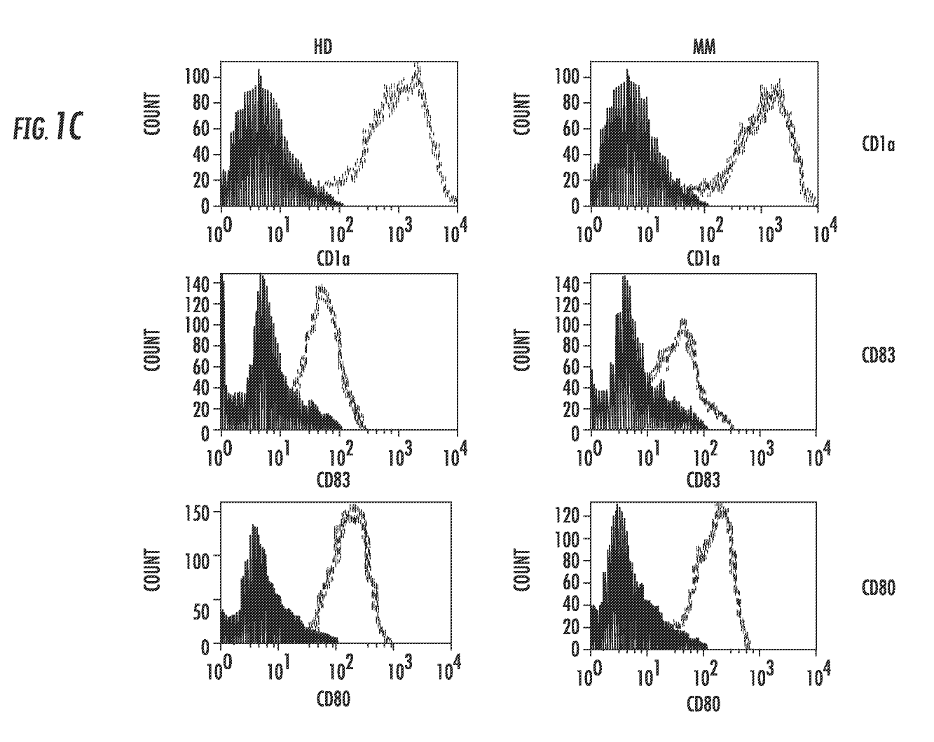

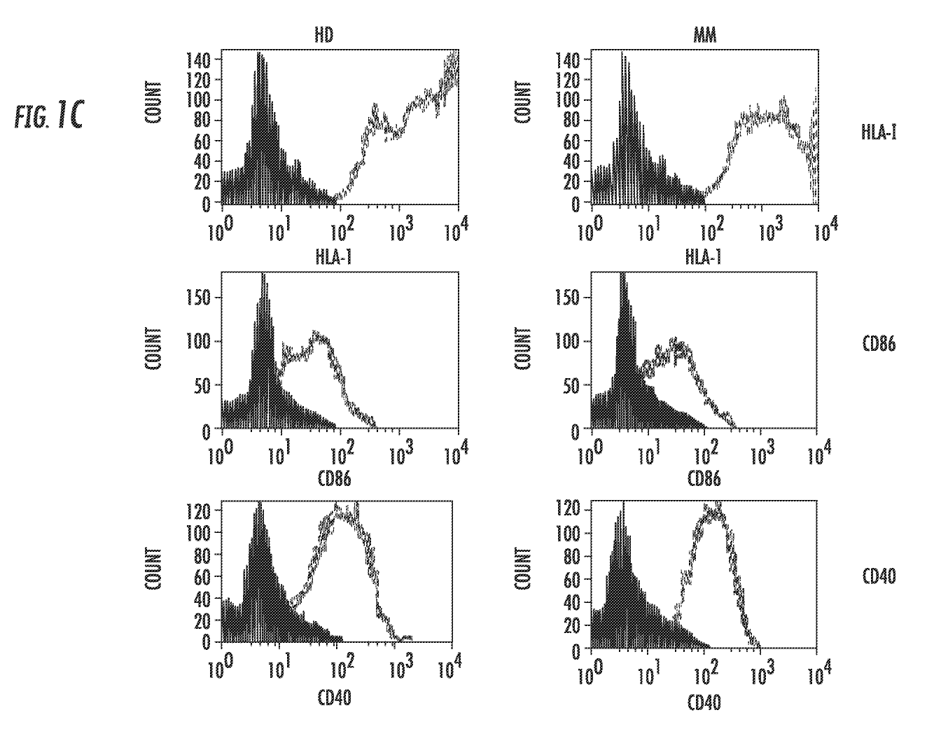

To evaluate MM patients' immune response, MM patients' DCs were pulsed with cancer cell lysates and co-cultured with autologous PBMCs. The experimental approach is illustrated in FIG. 1A. MM plasma cells were isolated from bone marrows of MM patients using an immuno-magnetic bead depletion method based on the MM surface phenotype: CD38⁺CD138⁺CD56⁺CD45⁻. The purity of the plasma cells after enrichment was 96-98% as confirmed by surface marker staining and flow cytometry analysis (FIG. 1B, top panel: before selection, and bottom panel: after selection).

Because DCs and their precursors in cancer patients may be immune compromised, the phenotype of ex vivo derived DCs from MM patients was assessed by preparing monocyte-derived DCs using a 5-day culture protocol as previously described (Chen et al., Retroviology 1:37, 2004). The immature DCs were treated with TNFα and LPS to induce maturation. Mature DCs from five healthy donors (HD) and five MM patients (MM) were analyzed with fluorescent antibodies specific for DC markers CD1a, CD83, CD80, HLA-I (-ABC), CD86, CD40, and HLA-DR. FIG. 1C illustrates the representative DC phenotypes. Although minor variations were observed, there was no significant difference in surface marker phenotype between the two groups.

### Expansion of CD4⁺CD25^{high}FoxP3^{high} Treg Cells by MM Cell Lysate-Pulsed DCs

For the analysis of the MM-specific immune response, immature DCs of MM patients were pulsed with cell lysates prepared by multiple freeze-thaws. The specimens for comparison included autologous MM cells (DC/MM), autologous normal PBMC (DC/PBMC) and the allogeneic EBV-transformed B cell line (DC/BCL). To examine the effect of the MM cell lysates on DC immunity against other antigens presented by the same DCs, immature DCs were infected with LV-LacZ, which encodes a highly immunogenic bacterial β-galactosidase protein, for 16-24 hours before exposure to MM cell lysates (DC-LV-LacZ/MM). As a control, immature DCs were infected with LV-LacZ, and directly induced into maturation (DC-LV-LacZ).

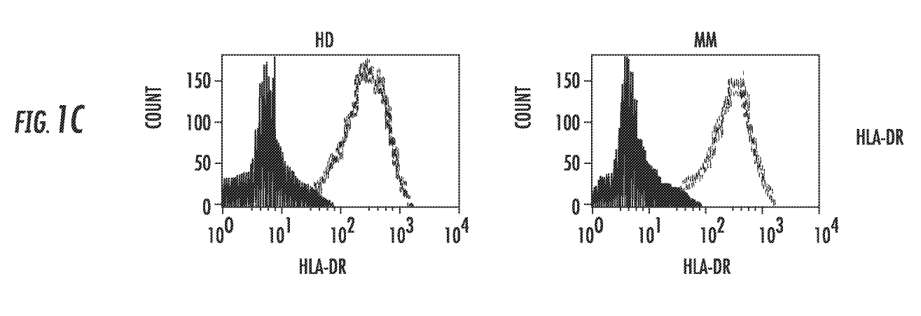

To determine the differentiation fate of antigen-specific T cells, DCs were co-cultured with non-adherent autologous PBMCs at a ratio of 1:20 for 14 days before the resulting T cells were re-stimulated with the same antigen-treated DCs. TNFα and IFNγ producing CD4 and CD8 T cells were detected by intracellular and surface antibody staining The results are summarized in FIG. 2A. It was observed that the CD4 and CD8 T cell activation functions of DCs were significantly suppressed when MM cell lysates were used to pulse the DCs (DC/MM and DC-LV-LacZ/MM versus DC/BCL and DC/LV-LacZ). The results showed that both CD4 and CD8 T cell responses were affected as illustrated by the expression of T cell activation cytokines TNFα and INFγ.

To examine the Treg response to different antigens, CD4 and CD25 positive T cells with the expression of FoxP3, an important transcription factor indicative of Treg activity, were gated for analysis. FIG. 2B clearly shows an increase in CD4 T cell population with CD25^{high} and FoxP3^{high} phenotype when the cells encountered the MM cell lysate (DC/MM, mean fluorescence index 8.09 vs. 2.64, 2.83 and 2.95 of DC alone, DC/PBMC and DC/BCL, respectively), with significant statistical difference (bottom panel). Thus, the MM cells selectively induced CD4⁺CD25^{high}FoxP3^{High} Treg cell expansion.

### Isolation and Expression of MM-Specific Idiotype Immunoglobulin (MM Id-Ig) Gene

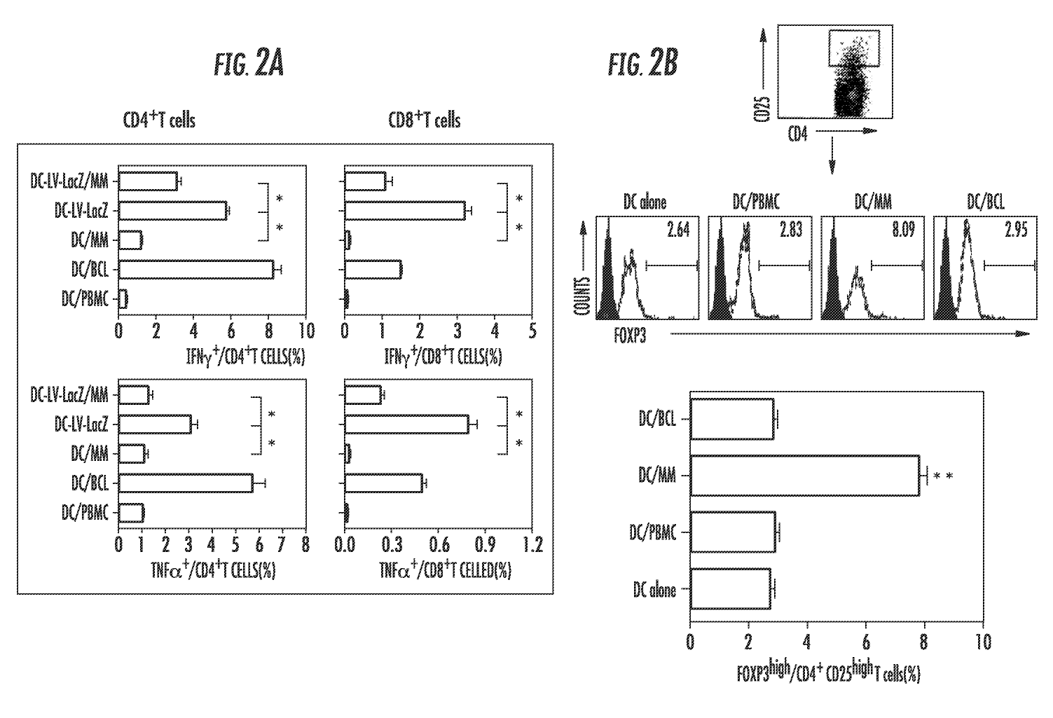

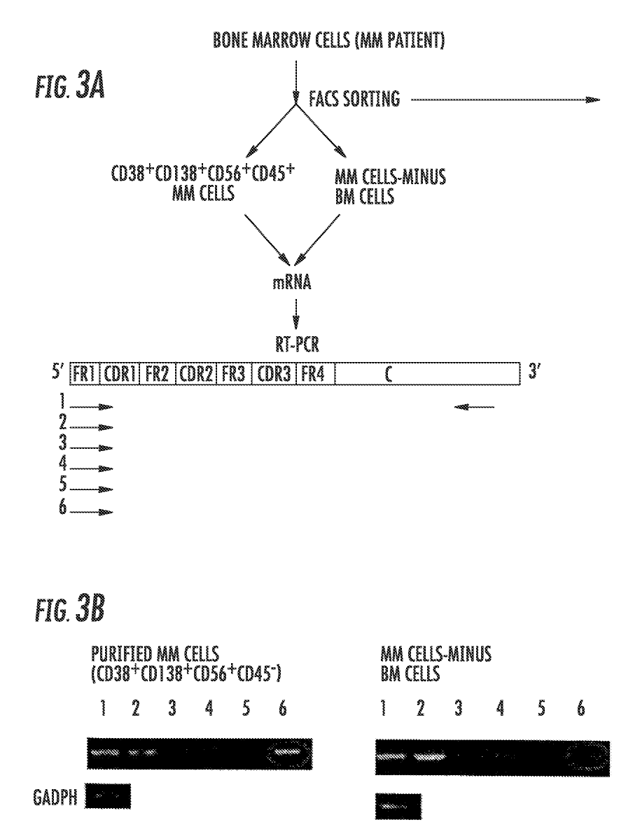

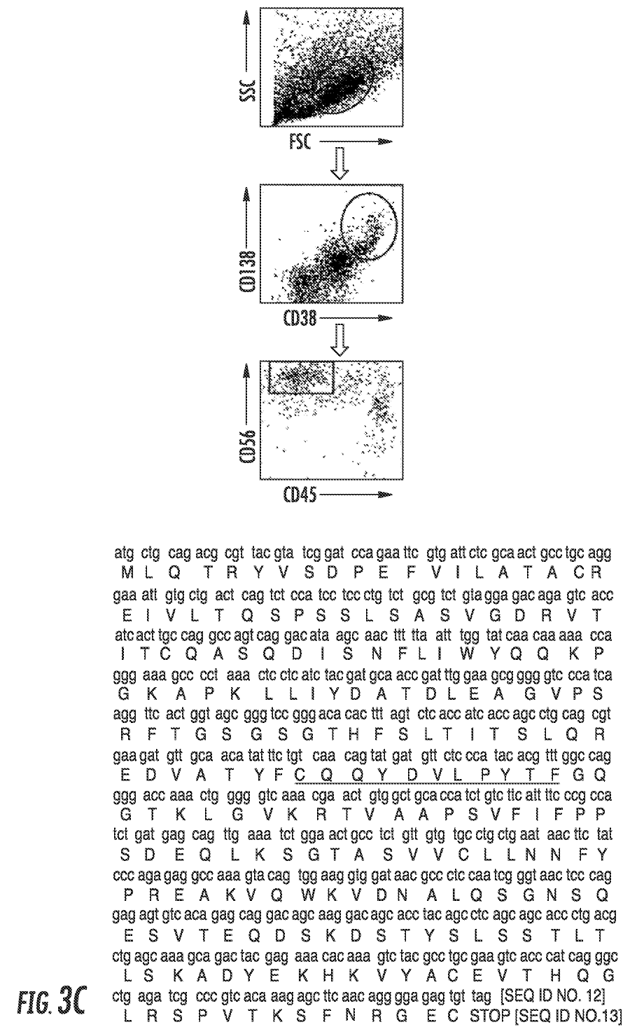

The activation of Treg cells by tumor cell lysate-pulsed DCs may be induced by multiple cancer-specific antigens. In order to see if this effect is specific to the MM-specific Id-Ig antigen, a kappa-chain specific MM patient's bone marrow cells were FACS-sorted based on the MM phenotype, CD38⁺CD138⁺CD56⁺CD45⁻ (right panel, FIG. 3A) and the RNA was harvested for cDNA synthesis. Six 5′ V-region primers and a common 3′ C-region primer were used to amplify the specific MM kappa gene (primer locations depicted on the kappa Ig gene diagram, FIG. 3A). The result shows that the sixth primer pair revealed a discordant amplification after PCR between MM+ and MM− cDNAs with a stronger signal for the MM+ cells (highlighted in red, FIG. 3B). Analysis of multiple cDNA clones using the amplified fragments generated the same CDR3 sequence, which suggests that they were derived from a clonal plasma cell (underlined region of the entire open reading frame of the specific MM kappa gene, FIG. 3C).

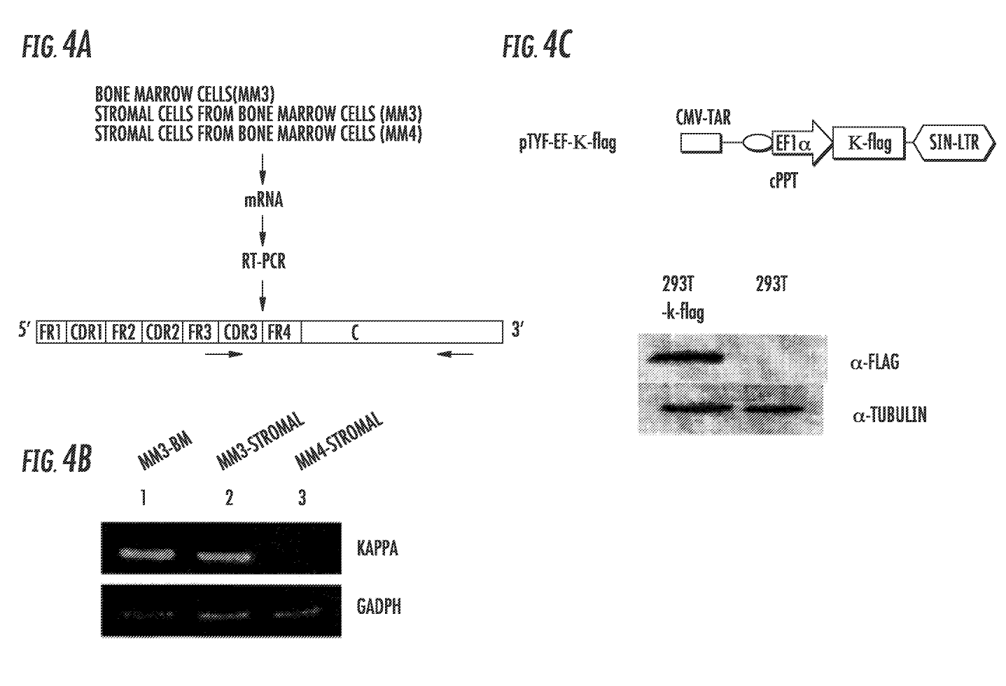

To confirm this result, an oligo-primer specific for the CDR3 sequence of the Id-Ig gene from patient MM3 was designed and used to amplify cDNAs from different sources of MM cells (FIG. 4A). Because it was noted in earlier studies that MM patients' bone marrow stromal cells continue to express high levels of MM-specific surface markers and idiotype Ig proteins, both bone marrow cells and bone marrow stromal cells were used for this analysis. A positive band was amplified from the corresponding MM3 patient's bone marrow cDNA (MM3-BM, lane 1, FIG. 4B) as well as the corresponding bone marrow-derived stromal cell cDNA (MM3 Stromal, lane 2), but not from a different MM patient's (MM4) bone marrow-derived stromal cell cDNA (MM4 Stromal, lane 3).

To ectopically express this Id-Ig gene in the patient's DCs, the MM kappa cDNA was cloned into a lentiviral vector (pTYF-EF) under the control of a strong EF1α promoter. For the detection of the MM kappa gene expression, the cDNA was fused with a C-terminal Flag tag (pTYF-EF-k-flag), and its expression was confirmed by Western analysis using an anti-Flag antibody (FIG. 4C, shown with an internal expression control of α-tubulin).

### MM Id-Ig Displays Low Immunogenicity but Induces a Specific Treg Cell Response

After confirming the MM Id-Ig gene expression by LV, the immunogenicity of the MM Id-Ig was assessed by infecting immature DCs 24-48 hr before maturation with the Id-Ig gene LV vector or a control vector LV-LacZ (DC-LV-Kappa and DC-LV-LacZ). A positive control was set up by pulsing immature DCs with the memory antigen tetanus toxoid (TT) for 4 hr before maturation (DC/TT).

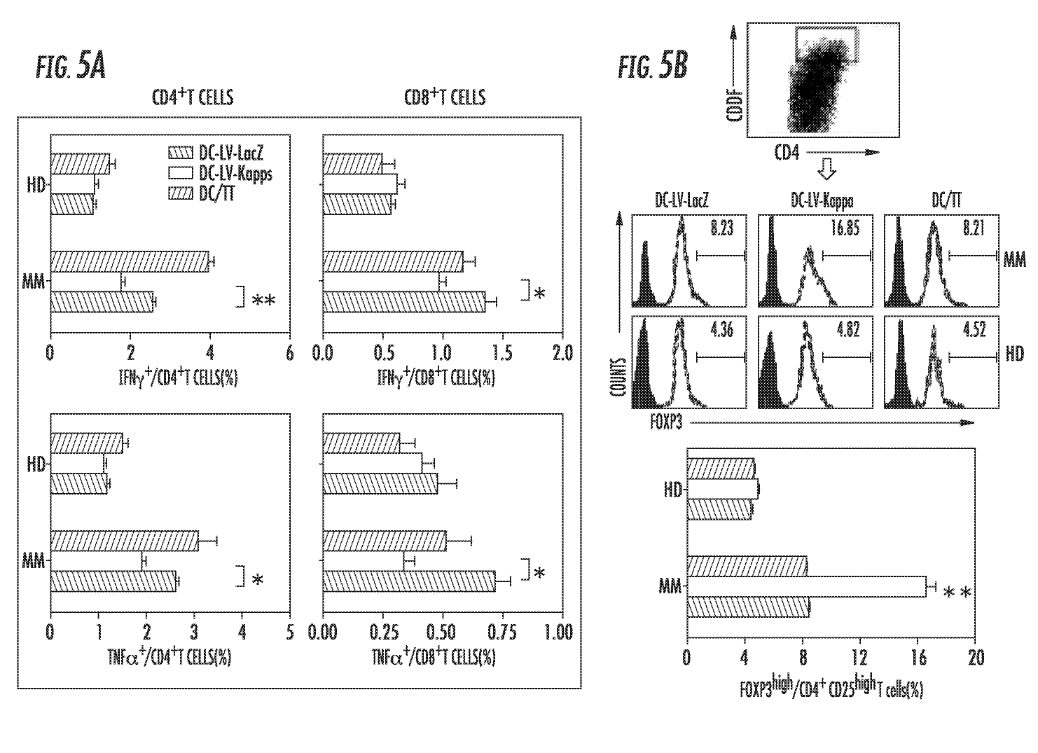

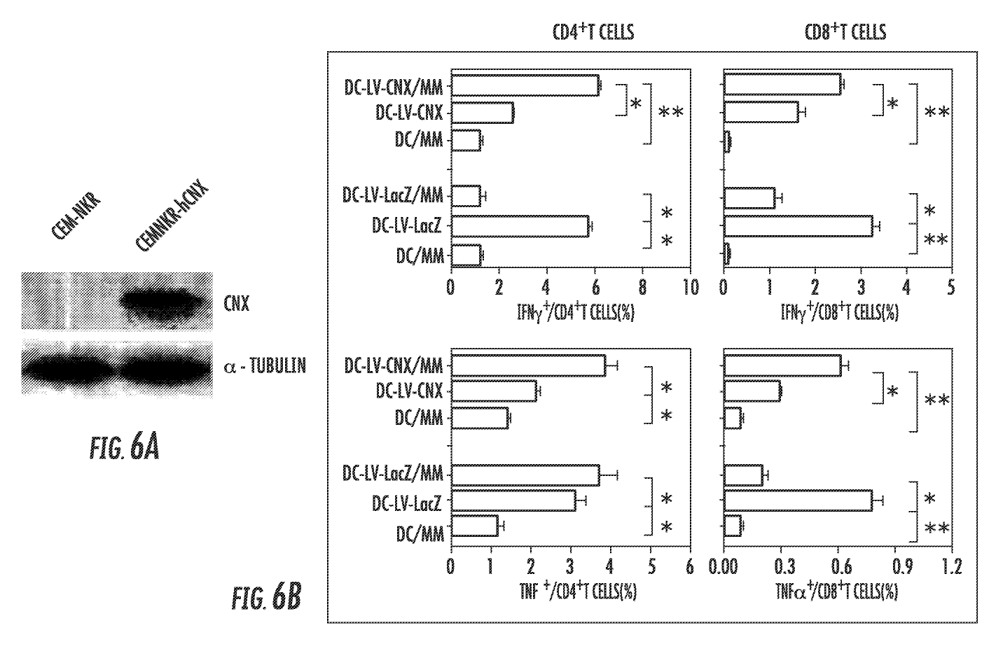

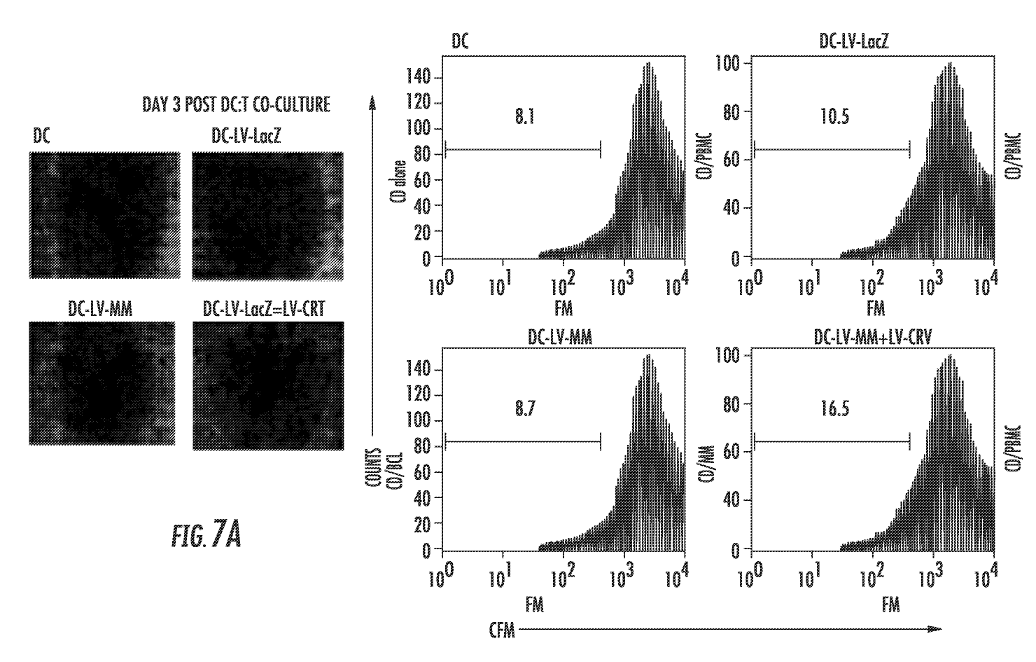

The DCs were co-cultured with T cells for 14 days and the T cell activation function was examined through multi-color surface marker and intracellular cytokine staining as described above. For healthy donors (HD), there was no difference between DC-LV-Kappa and DC-LV-lacZ in the CD4 and CD8 T cell response (FIG. 5A). In contrast, for MM patients, DC-LV-Kappa induced less CD4 and CD8 T cell response than did DC-LV-LacZ as illustrated by intracellular analysis of TNFα and IFNγ. (FIG. 5A, shown with significance analysis). This result is similar to that of the MM cell lysate-pulsed DCs.

Further analysis of CD4⁺CD25⁺ and FoxP3 Treg cells showed that DC-LV-Kappa, but not DC-LV-LacZ or DC/TT, up-regulated FoxP3 expression in the CD4⁺CD25^{high} T cells in the corresponding MM patients (MFI of 16.85 for DC-LV-Kappa, vs. 8.23 and 8.21 for DC-LV-LacZ and DC/TT, respectively, in the MM patient, p<0.001). In contrast, no significant difference was found for healthy donors (FIG. 5B).

### LV-CNX-Transduced DCs Enhance the MM-Specific CD4 and CD8 T Cell Response

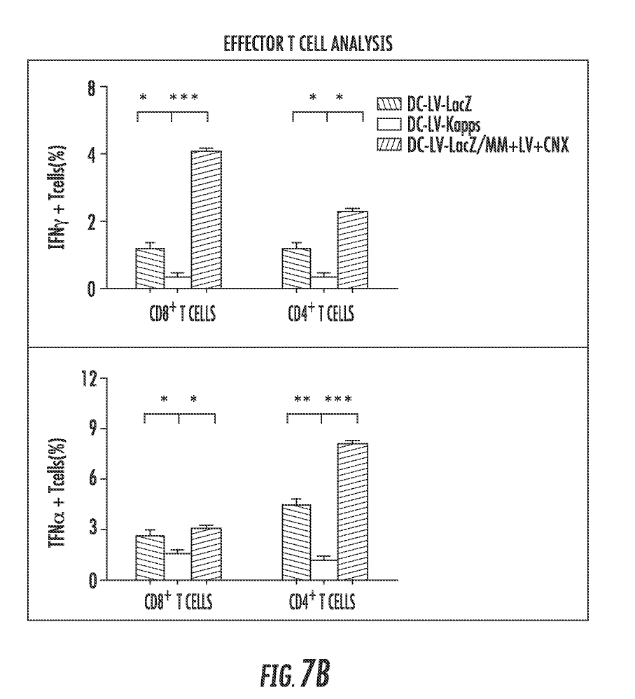

The above results illustrate that both the MM cell lysates as well as the MM-specific Id-Ig Ag could not induce an effector T cell response but instead, promote a strong Treg cell response. In an attempt to overcome this immune suppression, the antigen presentation functions of the MM patients' DCs were modified. The effect of calnexin on MM patients' DC and T cells by LV-mediated overexpression of calnexin in patients' DCs was investigated. The human calnexin cDNA was cloned into lentiviral vector LV-CNX, which was placed behind a strong EFla promoter. As CNX is universally expressed in most cell types, a calnexin-defective cell line, CEM-NKR, was used to verify the expression of LV-CNX. Western analysis detected high expression of calnexin in the CEM-NKR cells after LV-CNX transduction (FIG. 6A).

To assess the effect of LV-CNX, immature DCs were transduced with LV-CNX (DC-LV-CNX/MM) and LV-LacZ (DC-LV-LacZ/MM) as a control vector and pulsed with MM lysates. After maturation, the DCs were cocultured with autologous non-adherent PBMCs at a ratio of 1:20 for 14 days. The IFNγ and TNFα response of the T cells upon re-stimulation with antigen-specific DCs was analyzed by multicolor intracellular and surface staining. The results confirmed previous findings that MM lysates induced immune suppression of both CD4 and CD8 T cells (DC-LV-LacZ/MM vs. DC-LV-LacZ, FIG. 6B). Importantly, LV-CNX effectively reversed this trend (FIG. 6B, DC-LV-CNX/MM vs. DC/MM). However, analysis of the LV-CNX effect on Treg cells revealed no reduction in the number of CD4⁺CD25^{high}FoxP3^{high} T cells generated after coculture with the DC-LV-CNX/MM.

### LV-CNX Enhances the MM Id-Ig-Specific DC Immunity

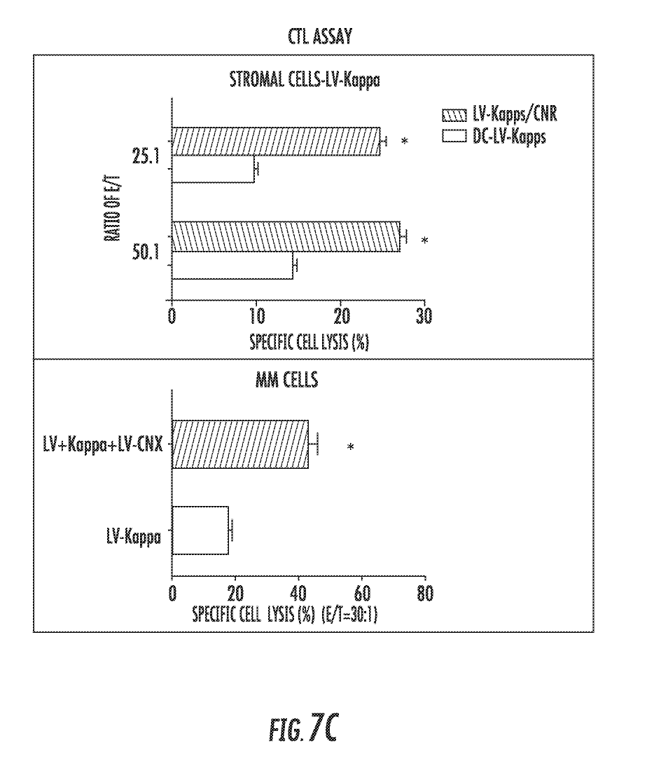

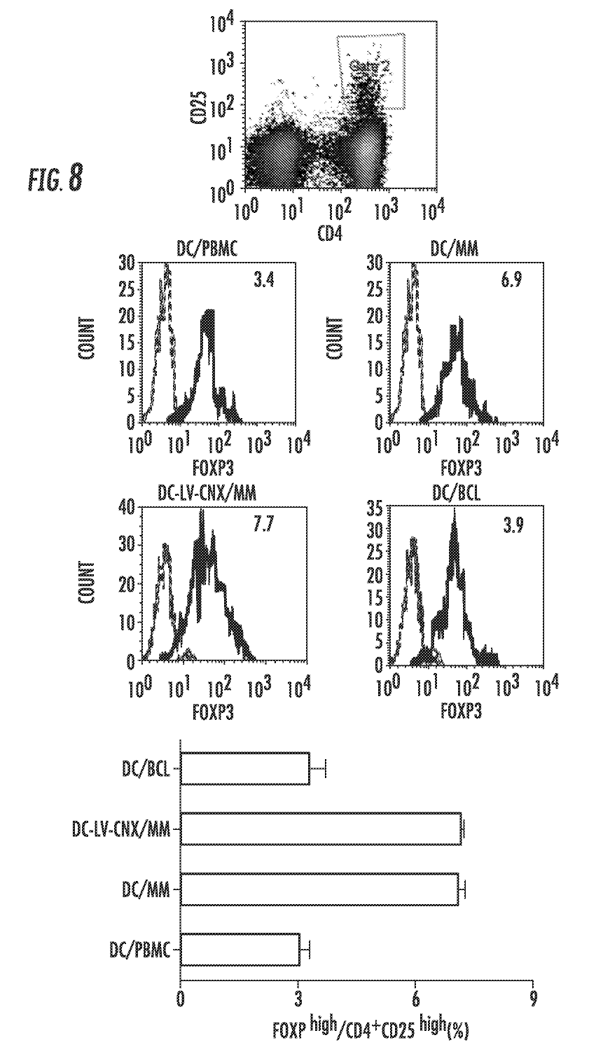

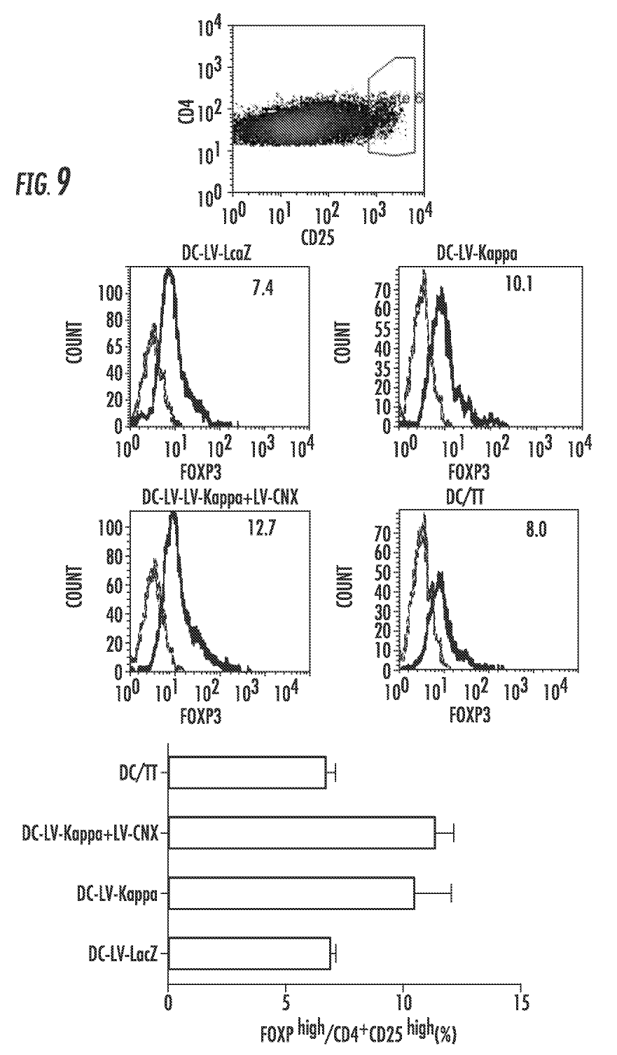

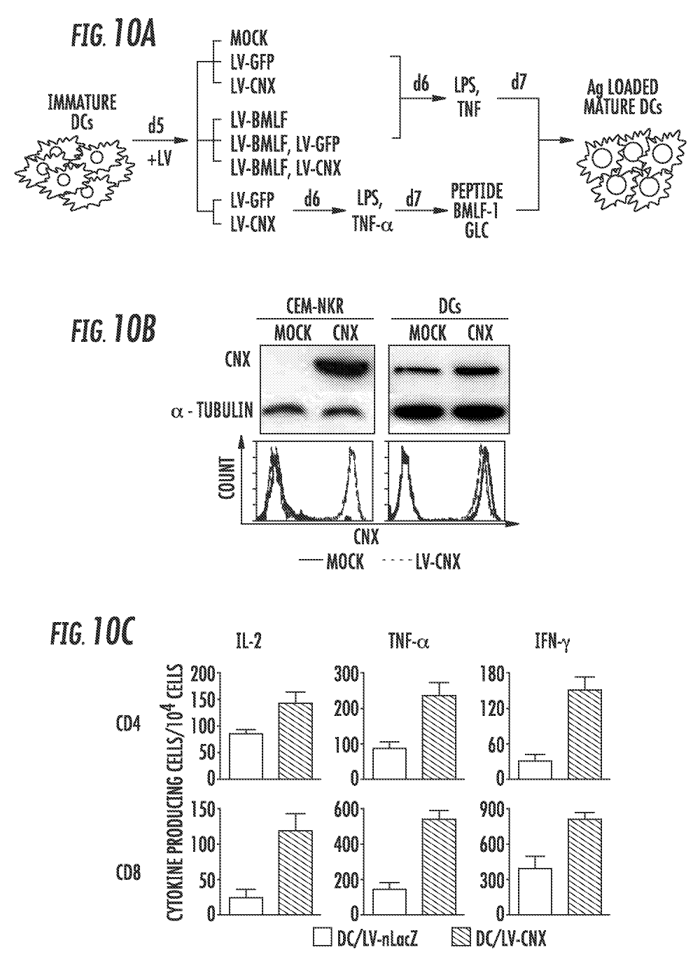

The above study demonstrates that MM patients' DCs, when transduced with LV-CNX, can effectively up-regulate MM-specific CD4 and CD8 T cell responses. A rapid T cell expansion was observed within three days of the DC and T cell co-culture when LV-CNX-DCs were used (FIG. 7A, clusters of expanded T cells). The rapid T cell expansion was verified by CFSE analysis of cell division. Before DC and T cell coculture, the T cells were pre-stained with CFSE; the intensity of CFSE in the T cells would decrease with increased T cell division. After three days in the coculture, the CFSE intensity of the LV-CNX DC coculture group was markedly decreased compared to those of the three other groups, indicating an increased rate of T cell proliferation in that specific group (FIG. 7A, right panel).

To determine whether CNX could overcome the tolerance of MM-specific antigens (MM-Id-Ig), the specific Id-Ig gene was isolated and constructed into LV. The corresponding MM patients' immature DCs were transduced 24-48 hr before maturation with the LV-Id-Ig (LV-Kappa), LV-Kappa plus LV-CNX (DC-LV-Kappa+LV-CNX), LV-Kappa alone (DC-LV-Kappa) or LV-LacZ alone (DC-LV-LacZ). The mature DCs were co-cultured with autologous non-adherent PBMC for 14 days, and the Ag-specific T cell response was examined by ICCS for IFNγ and TNFα secreting T cells. The result showed an enhancement in both CD4 and CD8 effector T cell activities when DCs were co-transduced with LV-CNX (FIG. 7B). While LV-CNX enhanced cytokine production of both CD4 and CD8 T cells, the number of CD4⁺CD25^{high}FoxP3^{high} Treg cells between the DC-LV-Kappa+LV-CNX and the DC-LV-Kappa alone was not significantly changed.

To directly correlate the immune effector function to anti-cancer immunity, the CTL activity of the MM-specific T cells was examined using a non-radioactive FATAL assay. DC-LV-Kappa and DC-LV-Kappa+LV-CNX cocultured T cells were re-stimulated with DC-LV-kappa for 5 days. The effecter cells were incubated with target cells including primary MM cells isolated with magnetic beads, autologous stromal cells infected with LV-Kappa (Stromal cells-LV-Kappa) or control target cells. For primary MM cells and stromal cells-LV-Kappa, the control target cells were autologous BCL and stromal cells transduced with LV-LacZ (Stromal cells-LV-LacZ), respectively. The cytotoxic effector function was measured as described in Materials and Methods. The results are summarized in the top panel of FIG. 7C. T cells derived from the DC-LV-Kappa+LV-CNX co-culture killed target cells with increased activity and specificity when compared with the control T cells at E/T ratio of 50:1 or 25:1 (P<0.03). To see if this CTL activity could target primary MM cells, autologous MM cells were isolated from the patient's bone marrow using specific antibody-conjugate magnetic beads. Autologous EBV-transformed B cells served as control cells. The result showed that the T cells from the coculture of DC-LV-Kappa+LV-CNX killed the MM target cells with increased activity (>40% of specific cell lysis at an E/T ratio of 30:1) as compared with the T cells from the DC-LV-Kappa co-culture (<20%, P<0.02, FIG. 7C bottom panel). The above studies clearly illustrate that supraphysiological expression of CNX in DCs enhances cytokine production and cytotoxic effector function of co-cultured T cells with specificity to kill primary MM cells.

### Example 2

### Potent Expansion of High-Avidity CTLs with Central Memory Phenotype by Lentiviral Modification of DCs Supraphysiologically Expressing Calnexin

DCs supraphysiologically expressing CNX engineered by LVs rapidly and efficiently sensitized antigen-specific T cells. Immunophenotype and microarray analyses of LV-CNX-engineered DCs showed increased expression of molecules related to Ag presentation and cell-cell adhesion. The CNX-DC-primed T cells exhibited increased functional avidity maturation and CCR7 expression. Functional array analysis of peptide-tetramer-purified Ag-specific T cells revealed upregulation of costimulatory molecules belonging to the TNF receptor superfamily. This increased T cell immunity was translated into therapeutic efficacy in a murine tumor model and resulted in an enhanced ex vivo cancer patients' anticancer immune response.

### Methods

### Human Monocyte-Derived DCs and T Cell Culture

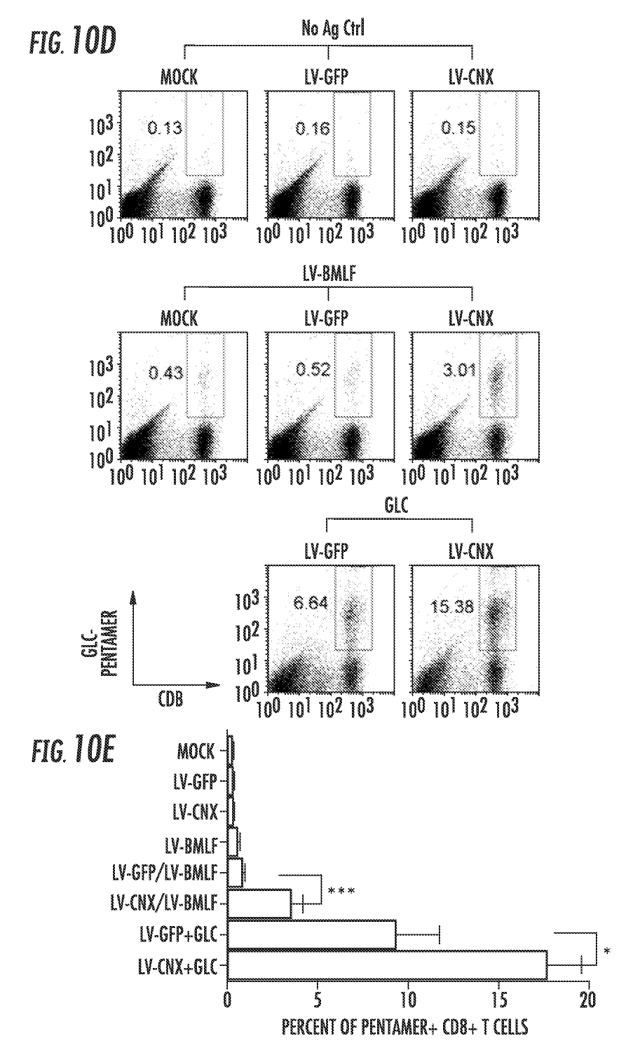

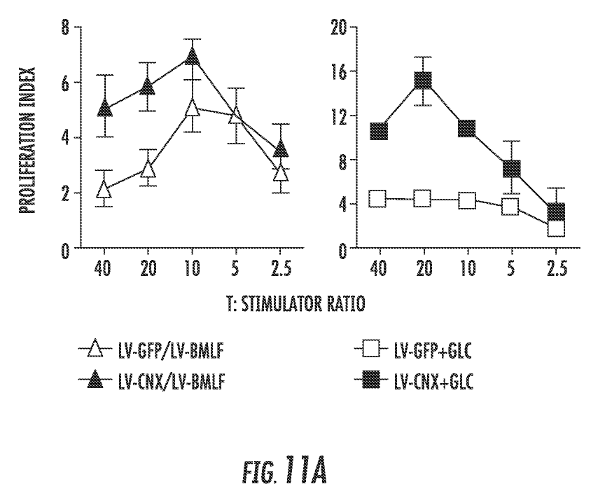

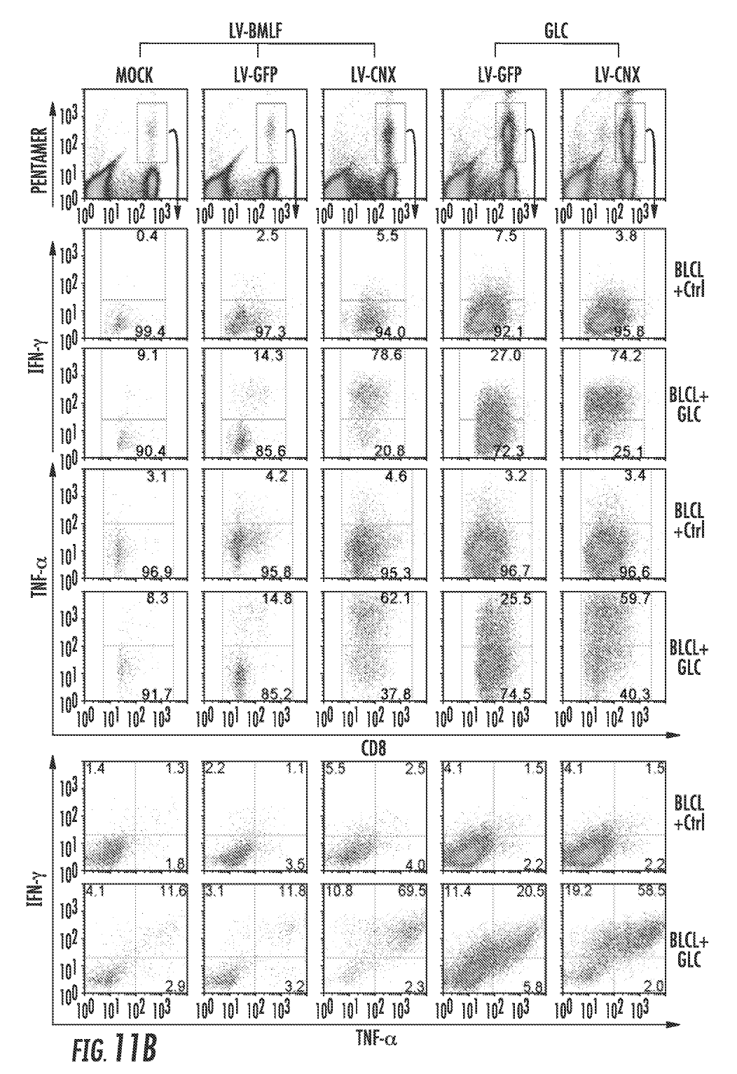

Blood samples were from healthy donors (Civitan Blood Center, Gainesville, Fla.) or patients with cervical carcinoma approved by Institutional Review Board (IRB) of University of Florida. Immature monocyte-derived DCs and total T cells were from peripheral blood mononuclear cells. Mononuclear cells isolated by Ficoll-Hypaque density centrifugation (Sigma-Aldrich, St. Louis, Mo.) were enriched for monocytes by plastic-adherence and immature monocyte-derived DCs were derived by culture in 50 ng/ml of granulocyte-macrophage colony-stimulating factor (GM-CSF) and 25 ng/ml of IL-4 (eBiosource International, Inc. Camarillo, Calif.) in serum-free AIM V medium (Invitrogen Corp. Carlsbad, Calif.). DCs were transduced by different LVs at day 5 as described before (Chen et al., Retrovirology 1:37, 2004), and loaded with peptides at day 7 as indicated in FIG. 10c. T cells were cocultured with differently modified mature DCs in a 24-well plate at a 20:1 ratio in AIM V medium containing IL-2 (12.5 U/ml), and IL-7 (10 ng/ml). At day 12 of coculture, T cells were restimulated or harvested for analysis as indicated. For T cell proliferation assay, T cells were labeled with CFSE (Molecular Probes, Eugene, Oreg.) and cultured in 96 well U-bottomed plates (Becton Dickinson) with GLC loaded irradiated BLCL at different ratios. Six days after stimulation, cells were harvested, and stained for GLC pentamer, and CD8 for FACS analysis.

### Antibodies and Reagents

Antibodies used for flow cytometry analysis were as follows: fluorescein isothiocyanate (FITC)-conjugated antibody to CD8α, CCR7, IFN-γ, HLA-I, CD86; phycoerythrin (PE)-conjugated CD4, PE-CD11c, CD83 and CXCR4; PE-Cy7-conjugated CD8α, CD62L, CD11b, CD11c, CD40 and CD80; allophycocyanin (APC)-conjugated, CD69, CD1a, CD54, and TNF-α (all from BD Pharmingen, San Diego, Calif.); FITC-CCR7 (R&D system), FITC-HLA-DR (Caltag); APC-DC-SIGN, and APC-CD28 (eBioscience). Isotype matched antibodies (BD Pharmingen) were used as controls. HLA-A2 EBV GLC pentamer was purchased from the ProImmune (Springfield, Va.) and HLA-A2 HPV16 E7 tetramer was purchased from the NIH Tetramer Core Facility (Atlanta, Ga.).

### Flow Cytometry, Intracellular Cytokine Staining (ICCS) and Multimer Staining

Flow cytometry data was acquired on a FACSCalibur flow cytometer (Becton Dickinson). All staining was performed on ice using fluorescence conjugated antibodies as indicated. For intracellular cytokine staining, T cells were washed and re-stimulated as indicated for 5 hr, with Brefeldin A (1 μg/ml) during the last 2.5 hr of culture. Cells were washed and permeabilized with the Cytofix-Cytoperm kit (BD Pharmingen), according to the manufacturer's directions and stained with FITC-IFN-γ, and/or TNF-α. Samples were resuspended in PBS containing 2% formaldehyde until analyzed by FACS. For pentamer staining, resting T cells were stained with PE-labeled pentamer for 12 minutes at room temperature, followed by FITC- or PECy7-labeled anti-CD8 for 30 minutes on ice. For tetramer staining, cells were stained with FITC-labeled anti-CD8 and PE-labeled tetramer for 30 minutes at room temperature. Data are analyzed using FlowJo.

### Fluorometric Analysis of T-Lymphocyte Antigen-Specific Lysis (FATAL Assay)

The CTL assay was based on a non-radioactive FATAL assay described before (Sheehy et al., J Immunol Methods 249:99-110, 2001), with the following modifications. BLCL pulsed with control or GLC peptide were used as target cells. Target cells were first labeled with PKH-26 (Sigma, St. Louis, Mo.) followed by CFSE staining The double labeled target cells were dispensed in duplicate at 5×10⁴ cells/well into U-bottom wells. T cells were added at various E:T ratios and mixed with the target cells. After 16 h incubation, the cells were harvested and fixed in 1% par formaldehyde in PBS and analyzed by FACSCalibur flow cytometer and the CELLQUEST program (BD). PKH-26 positive cells were gated and same cell numbers were acquired for each sample. The percentage of specific cell lysis in the target cell population was determined by the disappearance of the antigen-specific targets from the CFSE^{hi} population compared to the control targets in the CFSE^{hi} population. To calculate the percentage of the specific lysis the following equations were used: Percent survival=(mean CFSE^{hi} percent of test well/mean CFSE^{hi} percent of spontaneous release)×100. Percent specific lysis=100−% survival.

### Purification of Pentamer-Positive CTLs and Real-Time PCR Array Analysis

Total primed T cells were stained with PE-labeled GLC pentamer for 15 minutes in room temperature. Cells were washed and then incubated with anti-PE beads and pentamer positive T cells were isolated by positive selection (Stem Cell Technologies, Vancouver, BC) according to the manufacture's instructions. Isolated CTLs were restimulated in vitro for an additional five times and seven days after the last stimulation, cells were harvested for RNA extraction and cDNA synthesis. Real-time PCR array analysis was performed according to the manufacture's instructions (SupperArray Bioscience Corporation, Frederick, Md.).

### Tumor Therapy

Balb/c mice were inoculated with 1×10⁵ CT26-E6E7 tumor cells subcutaneously. Seven days later, mice were vaccinated with 2.5×10⁵ DC/LV-nLacZ, DC/LV-opiE6E7, or DC/LV-opiE6E7/LV-CNX weekly for 3 weeks (n=5 per group). The tumor size was measured over time using caliper and the mean tumor volume (in mm³) was determined. After 25 days splenocytes were harvested and analyzed for intracellular IFN-γ and TNF-α production.

### Statistical Analysis

Statistical analysis was performed with Student's t-test and Prism 4 software (GraphPad Software).

### Results

### Potent Expansion of Ag-Specific CD8⁺ T Cells by LV-CNX-DCS

Human day 5 immature DCs were generated from adherent PBMC-derived monocytes and transduced with different LV constructs (FIG. 10a). The expression of LV-CNX was demonstrated using a CNX-deficient cell line CEM-NKR (FIG. 10b), and upregulation of CNX in LV-CNX-transduced DCs was confirmed by immunoblot assay and flow cytometry (FIG. 10b). Because DCs are potent T cell activators, an MLR was first performed to determine whether upregulation of CNX would affect the T cell stimulation capacity of DCs. Allogeneic T cells were stimulated with DCs transduced with LV-nLacZ or LV-CNX and tested for effector cytokine production. The results showed that LV-CNX-DCs significantly increased expression of IL-2, IFN-γ, and TNF-α in both CD4 and CD8 T cells (FIG. 10c). Because LV-CNX engineered DCs increased allogeneic T cell response, LV-CNX-DCs were next examined in autologous T cell sensitization settings.

To determine the T cell priming efficiency of the LV-CNX engineered DCs, PBMC derived DCs were modified as illustrated in FIG. 10a and cocultured with autologous T cells. The Ag processing and presentation functions of DCs were evaluated by the analysis of T cells that recognized an HLA-A2 restricted epitope from Epstein-Barr virus (EBV) BMLF-1 protein (GLCTLVAML, abbreviated GLC). The GLC epitope was introduced into DCs either of two different ways, direct peptide loading or LV transduction. For the latter method, DCs were transduced with LV encoding a full-length BMLF-1 cDNA for endogenous Ag processing and presentation. After DC and T cell coculture for 12 days, GLC-specific T cells were detected using a GLC peptide-MHC pentamer and analyzed by flow cytometry. Increased expansion of GLC-specific CD8 T cells was observed when stimulated with Ag loaded LV-CNX-DCs (FIG. 10d, e). The effect of CNX was more prominent in LV-BMLF transduced DCs (˜5 fold, the LV-BMLF group) than peptide loaded DCs (˜2 fold, the GLC group), consistent with the roles of CNX in Ag processing and presentation pathways. These results show that the upregulation of CNX promotes the T cell sensitization capacity of DCs.

### Increasing Effector Functions of CTLs by LV-CNX-DCs

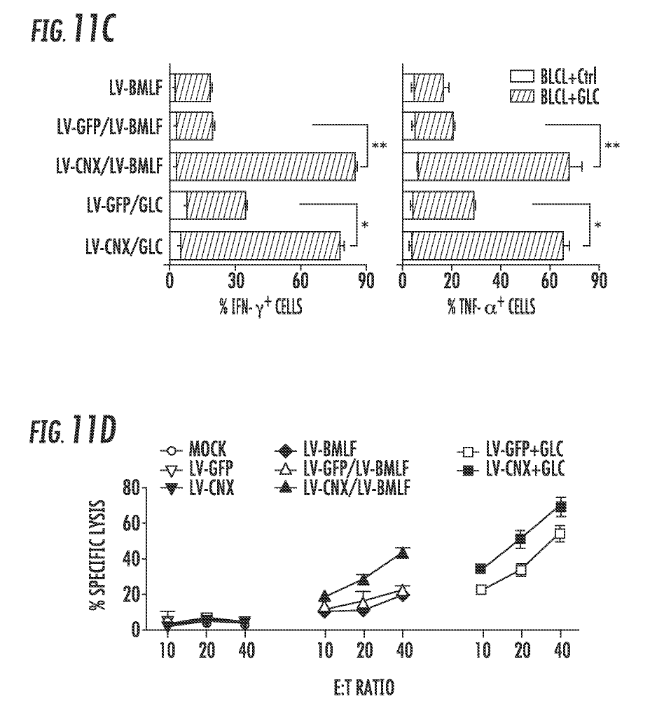

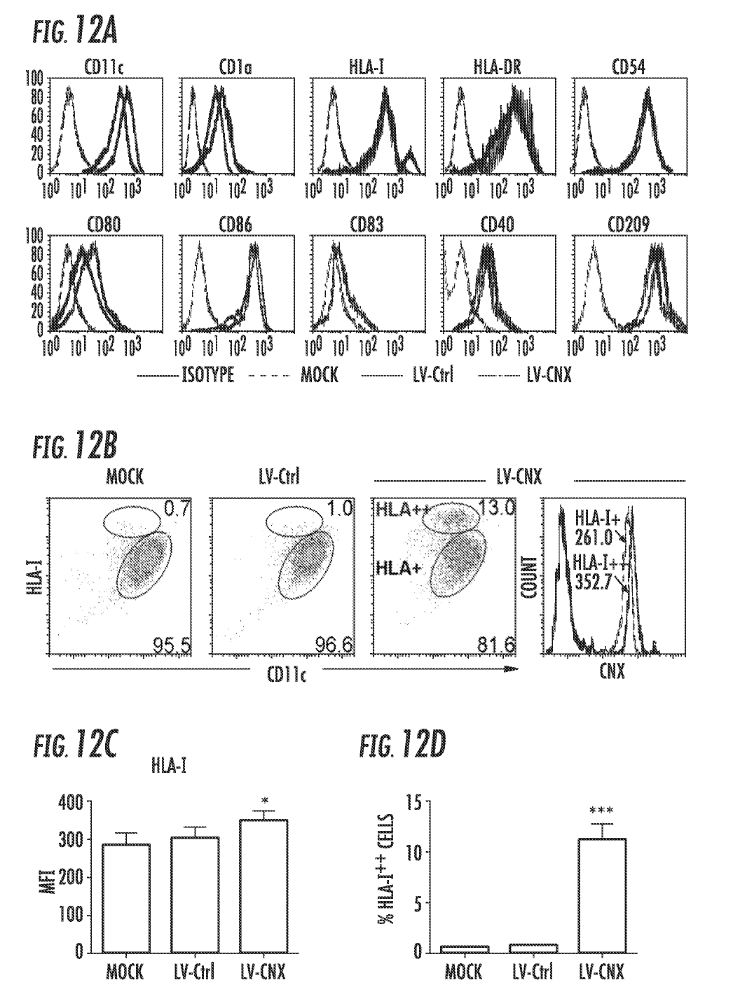

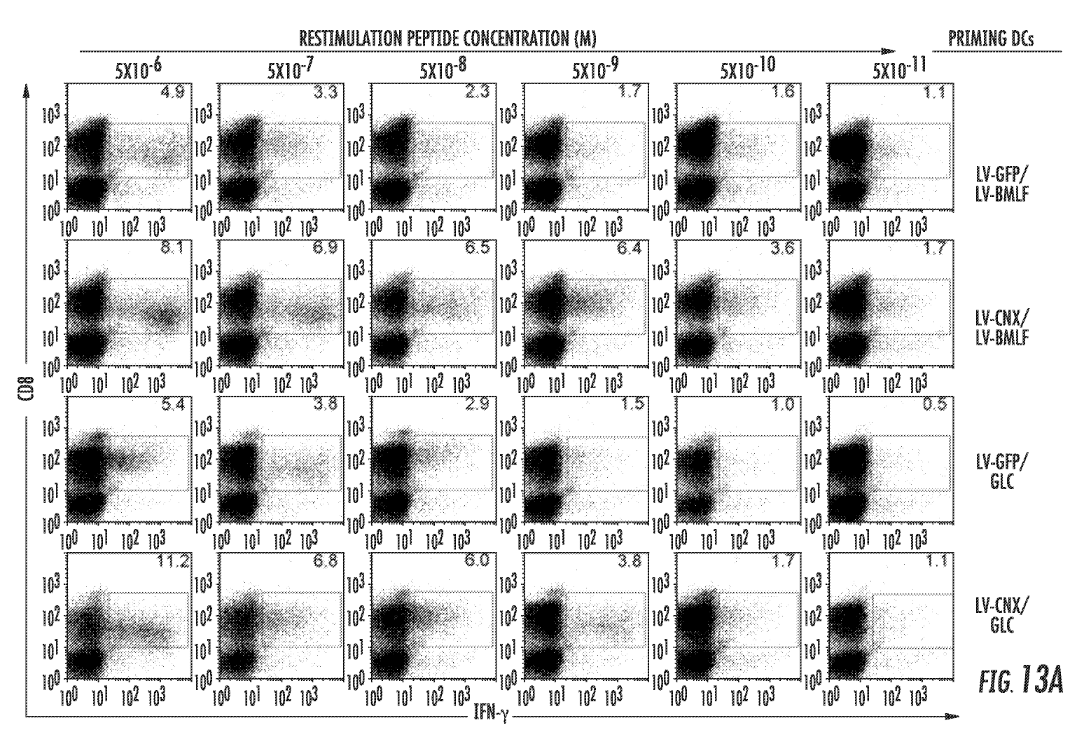

To determine whether the expanded CTLs maintain their effector functions, Ag-specific proliferation, cytokine production (IFN-γ and TNF-α), and cytolytic activities of the CTLs were examined. The proliferation ability of the Ag specific T cells was analyzed using a flow cytometry based method, which has been developed using the membrane-associated fluorescent dye CFSE to detect dividing cells through dilution in CFSE intensity. The sensitized T cells were labeled with the CFSE and stimulated with GLC peptide loaded BLCL at different ratios and cultured for 6 days. Ag-specific T cells were double-stained with fluorochrome-labeled GLC-pentamer and anti-CD8 antibody. This allowed analysis of the proliferation within a subset of T cells specific for a single epitope. Gated pentamer-positive CTLs were analyzed by Modfit software and the results are summarized in FIG. 11a. GLC-specific CTLs sensitized by CNX engineered DCs underwent increased cell divisions after GLC peptide or LV-BMLF stimulation.

Next, effector cytokine production of the T cells was compared using peptide-pulsed BLCL as stimulators. Total CTLs primed by CNX engineered DCs significantly increased the production of both IFN-γ and TNF-α (3˜4 fold), regardless the Ag loading methods (FIG. 11b). The flow cytometry profiles shown in FIG. 11b were obtained by gating the pentamer and CD8 double positive cells, and plotted against IFN-γ and TNF-α intracellular staining (y-axis). When stimulated with control peptide (Ctrl), few pentamer-positive cells spontaneously produced effector cytokines. The results showed that GLC-specific CTLs primed by CNX-DCs significantly increased both IFN-γ and TNF-α production (FIG. 11c). The increases observed were more significant when DCs were transduced with LV-BMLF (˜5 fold) than pulsed with the GLC peptide (˜2 fold).

To analyze the cytotoxic function of the GLC-specific T cells, FATAL (fluorometric assessment of T lymphocyte antigen specific lysis) assay was performed. BLCLs pulsed with control or GLC peptides were used as target cells and the lysis of target cells was indicated by the loss of cytoplasmic CFSE. Results from different donors reproducibly showed that T cells sensitized by the CNX-engineered DCs (i.e., CNX-DCs) exhibited increased cytotoxic activity (FIG. 11d). These results conclude that LV-CNX-DCs display increased effector priming functions including Ag-specific proliferation, cytokines production, and cytotoxicity.

### Immunophenotypic Analysis of LV-CNX-DCs

The observation that LV-CNX-DCs rapidly expanded large numbers of Ag-specific CTLs with enhanced functions prompted investigation of the underlying mechanisms. To this end, the surface marker phenotypes of mock, LV-Ctrl (LV-nLacZ) and LV-CNX transduced DCs were first compared. After maturation, all the DCs expressed typical DC surface markers, including CD11c, CD11b, CD1a, HLA class I/II (DR), CD40, CD80, CD86, CD83, CD54 and DC-SIGN (FIG. 12a and FIG. 17). In LV-CNX-transduced DCs, surface expression of CD11c, CD11b and CD1a (FIG. 17) was significantly downregulated. LV-CNX-DC consistently upregulated surface expression of HLA class I molecules (FIG. 12a-d). The overall surface expression of HLA-I was increased as analyzed by mean fluorescence index (FIG. 12c). A prominent population of CD11c⁺ cells with high expression levels of HLA-I (HLA-I⁺⁺) was detected in DCs transduced by LV-CNX (an average of 11.36% from six different donors), as compared with the mock or LV-Ctrl transduced cells (an average of 0.7-1%). Further analysis of intracellular CNX expression by flow cytometry in the HLA-I⁺ and HLA-I⁺⁺ cell populations showed that the expression levels of CNX were higher in the HLA-I⁺⁺ population (FIG. 12b). The effect of CNX on surface antigen epitope display by MCH-1 was further confirmed using specific monoclonal antibody to antigenic peptide-MHC-1 complex using mouse bone marrow-derived DCs.

### Increasing TCR Functional Avidity by LV-CNX-DCs

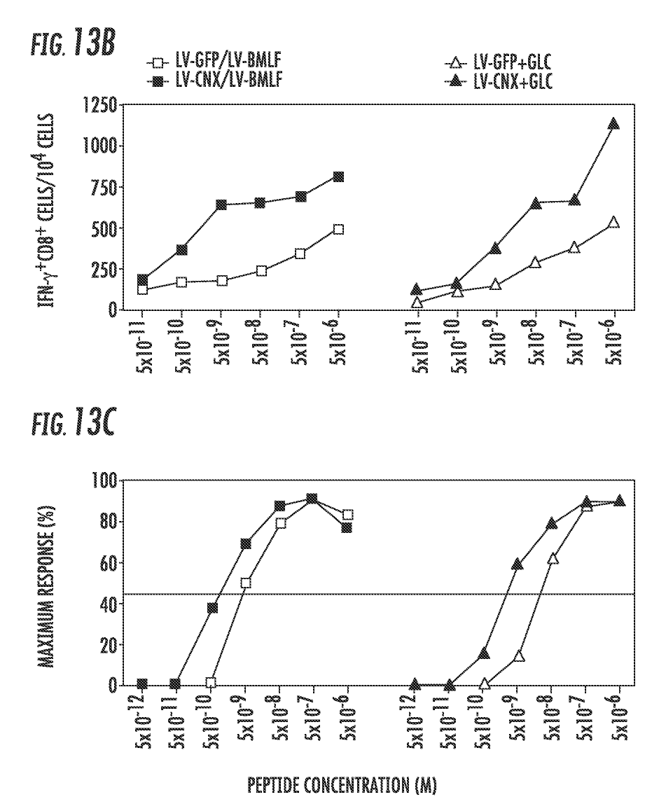

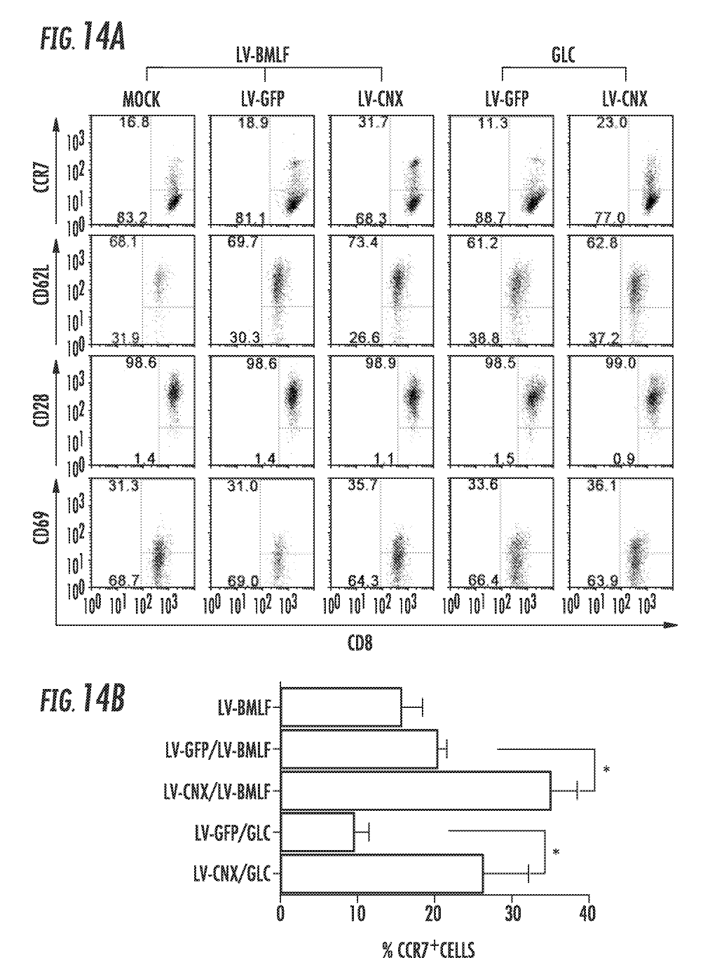

T cell functions and phenotype differentiation are influenced by the activation signals of DCs, and the functional avidity is an important factor to CTL quality. To explore whether LV-CNX-DC could affect CTL avidity, the Ag responsiveness of the expanded CTLs was first analyzed by measuring IFN-γ production to graded concentrations of the target peptide (GLC). T cells primed by DCs transduced with LV-CNX or control LV-GFP, either pulsed with GLC peptide or transduced with LV-BMLF1, were restimulated at day 12 with BLCL loaded with GLC peptide of graded concentrations. Six hours after restimulation, T cells were harvested for intracellular IFN-γ staining. As shown in FIG. 13a,b, CD8⁺ T cells primed by LV-CNX-DCs displayed increased IFN-γ responsive after GLC peptide stimulation. The functional avidity of GLC-specific CTLs was determined by quantifying the primed T cells with GLC-pentamer and intracellular IFN-γ production at different concentrations of restimulation peptide. The data are normalized to percentage of maximal cytokine responses as shown in FIG. 13c. The avidities, illustrated at 50% maximal cytokine response of CTLs to LV-CNX-DCs primed with BMLF and GLC peptide were 7.7×10⁻¹⁰ M and 3.1×10⁻⁹ M, respectively; whereas the avidities of CTLs to by LV-GFP DCs primed with LV-BMLF and GLC peptide were 4.1×10⁻⁹ M and 3.2×10⁻⁸ M respectively.

### Enhanced CCR7 Expression and Effector Function Profile of T Cells

To see whether CNX engineered DCs would affect T cell differentiation function, T cell surface markers for central/effector memory function including CCR7, CD62L, CD28, and CD69 were analyzed. FIG. 14 illustrates that Ag-specific CTLs (both LV-BMLF and GLC) primed by CNX engineered DCs displayed increased CCR7 expression, but not CD62L, CD28, or CD69. Thus, T cells sensitized by CNX engineered DCs appear to favor differentiation towards increased migration and central memory paradigm. The increased central memory T cell generation may also attribute to the significantly increased proliferation ability of CTLs.

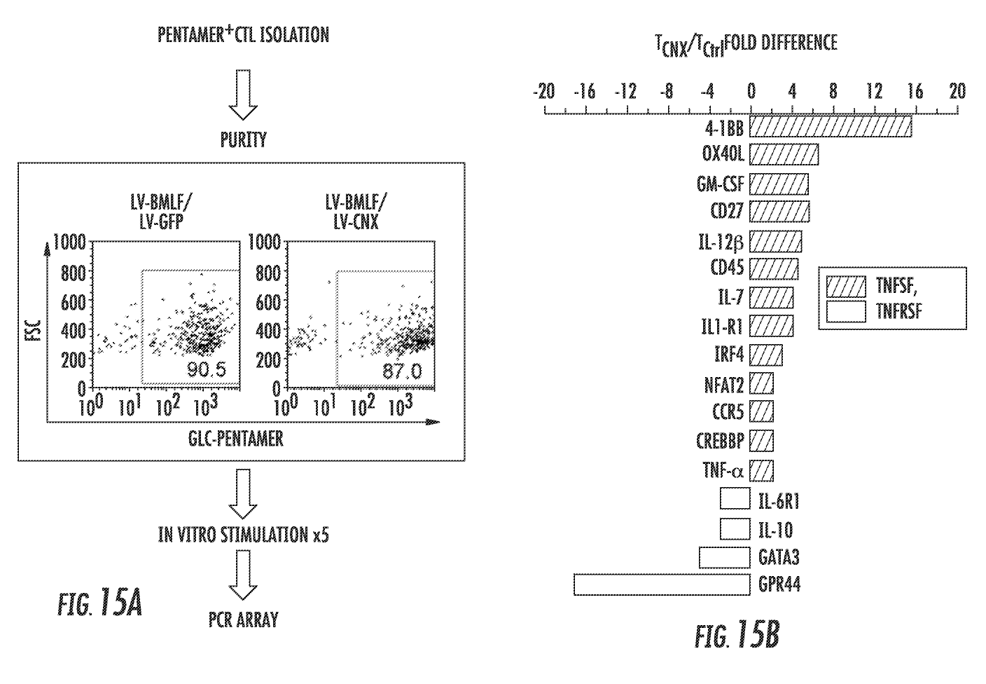

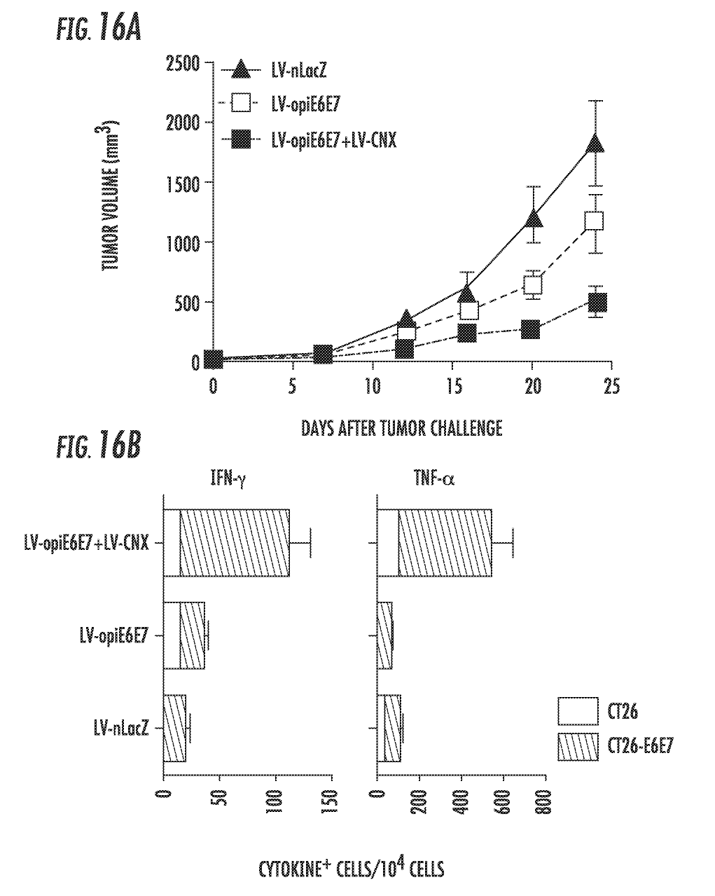

To examine the genetic profile related to T cell differentiation, real-time PCR array was performed designed for a focused panel of genes related to T cell differentiation. CTLs were cocultured with DCs cotransduced by LV-GFP/LV-BMLF (T_{Ctrl}) or LV-CNX/LV-BMLF (T_{CNX}). The primed Ag-specific CTLs were purified using PE-labeled GLC-MHC pentamer and anti-PE Ab magnetic beads and stimulated for five more rounds with BLCL pulsed with GLC peptide (FIG. 15a). RNA was extracted and reverse transcribed to cDNA and used for the PCR array reaction. The result showed that T_{CNX} upregulated the expression of genes involved in T cell activation and Th1 polarization and down-regulated genes involved in Th2 differentiation such as GPR44 and GATA3 (FIG. 15b). The expression levels of many costimulatory molecules belonging to TNF receptor superfamily (TNFRSF), such as 4-1BB (TNFRSF9), CD27 (TNFRSF7), and OX40L (TNFSF4) were upregulated in CTLs primed by CNX-DCs. Activation signals through these receptors have been shown to increase T cell proliferation, effector functions and memory generation. This result is consistent with the upregulated effector functions of the CTLs primed by LV-CNX-DCs.

### Increased Therapeutic Efficacy of CNX-DC Vaccine

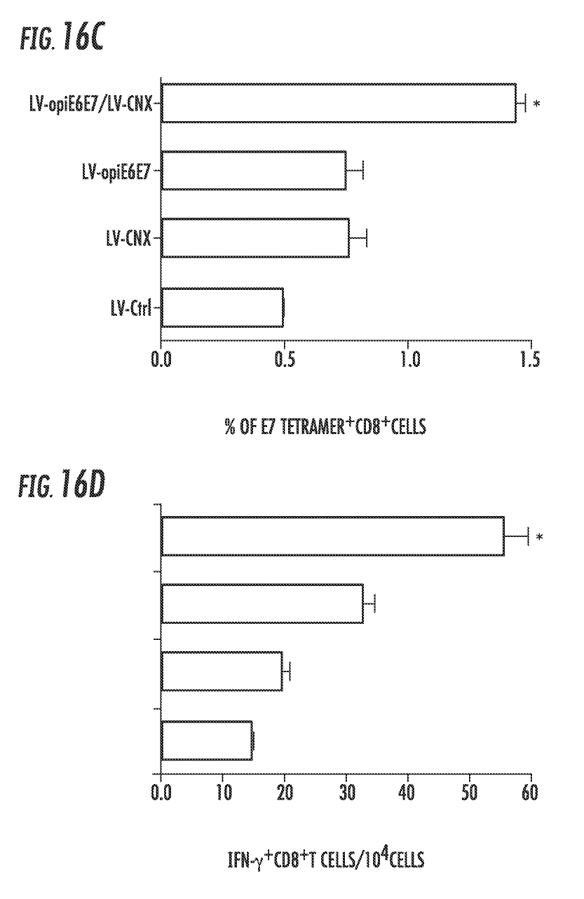

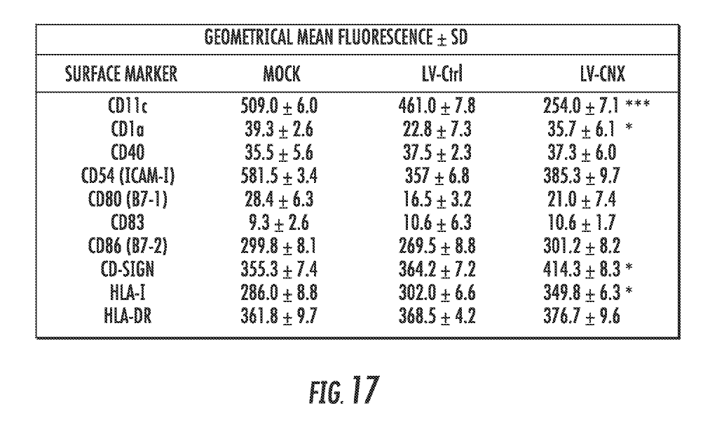

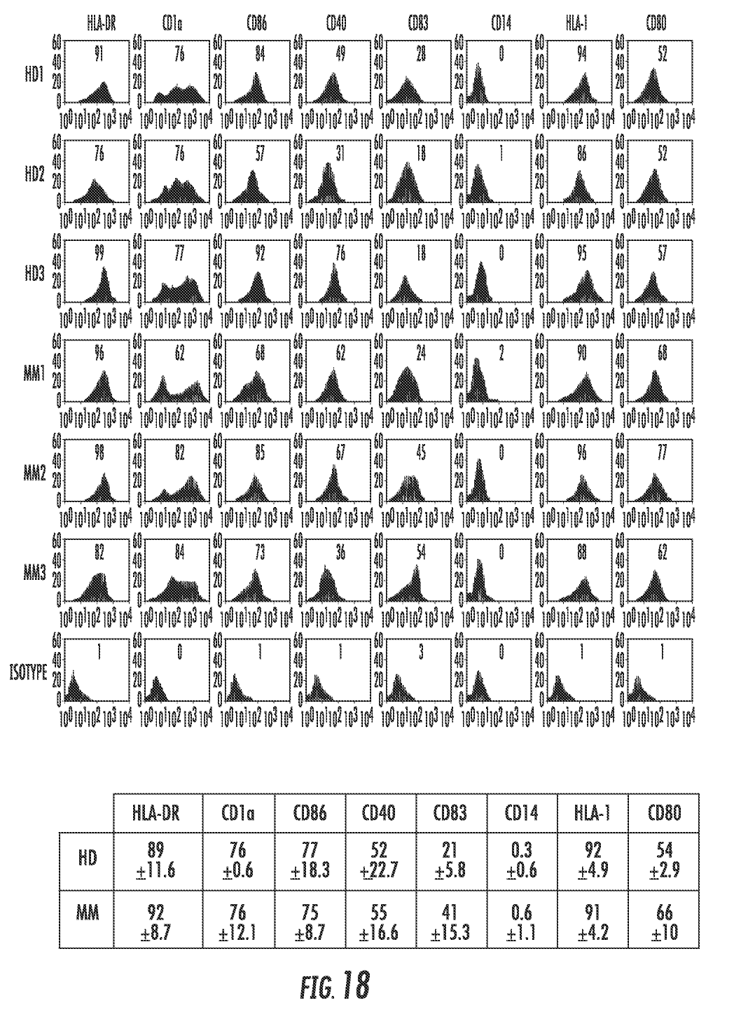

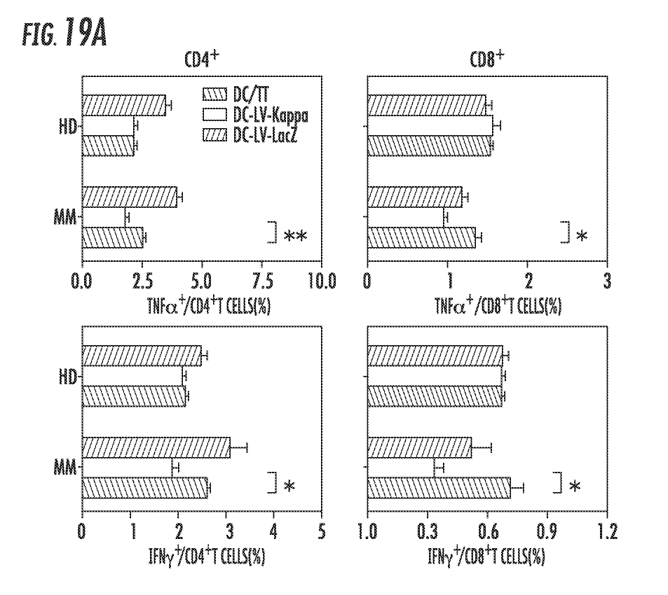

To determine whether the effects of CNX can be translated into therapeutic efficacy in vivo, a tumor therapy study was performed in an established subcutaneous mouse tumor model. The model tumor Ags were human papillomavirus 16 (HPV16) E6 and E7 proteins. Infection with HPV16 and HPV18 is highly associated with the development of cervical intraepithelial neoplasia and cervical carcinoma. The two early viral oncogenes, E6 and E7, are selectively retained and constitutively expressed in cancer cells and are therefore attractive immunotherapeutic targets. An LV encoding a codon-optimized E6E7 fusion gene (LV-opiE6E7) was constructed, which expresses higher levels of E6E7 compared with an unmodified construct. A Balb/c mouse colon cancer cell line, CT26, was transduced with LV-opiE6E7 to generate CT26-E6E7 tumor cells. Balb/c mice were implanted subcutaneously with 1×10⁵ CT26-E6E7 cells in their back and when tumors were palpable (day 7), they were subcutaneously vaccinated with LV-modified DCs (2.5×10⁵), including LV-nLacZ, LV-opiE6E7 and LV-opiE6E7+LV-CNX, followed by a second vaccination 7 days later. The tumor volume was measured every other day for 25 days. The LV-opiE6E7+LV-CNX-DC vaccine significantly reduced tumor growth in vivo (FIG. 16a). The LV-opiE6E7-DC vaccine group showed moderate reduction of tumor growth. To evaluate a tumor-specific immune response, splenocytes were harvested from the vaccinated mice and an E6E7-specific CD8⁺ T cell response was examined by intracellular IFN-γ and TNF-α staining As shown in FIG. 15b, LV-opiE6E7+LV-CNX-DC induced the highest IFN-γ/TNF-α production compared with the other two groups (LV-opiE6e7+LV-nLacZ).

The immune modulatory effect of CNX on DC: T cell coculture was further investigated under an ex vivo late stage cervical carcinoma patients' immune cell setting. The patients' T cells were primed by the autologous DCs transduced with mock, LV-CNX, LV-opiE6E7, or LV-opiE6E7/LV-CNX. At day 14 after coculture, HPV E7-specific T cells were detected using an HLA 0201-restricted E7 tetramer. It was evident that LV-opiE6E7/LV-CNX-DCs significantly increased the expansion of E7-specific T cells (FIG. 16c), with enhanced IFN-γ production (FIG. 16d). This was further confirmed in a separate study, wherein it was demonstrated that LV-CNX-DCs overcome the tolerance against multiple myeloma and induce a strong tumor-specific effector T cell response. These data suggest that LV-CNX-DCs help overcome tumor tolerance by optimizing T cell activation and increasing effector functions.

### Example 3

### Overcoming Immune Tolerance Against Multiple Myeloma with Lentiviral Calnexin-Engineered Dendritic Cells

The key to successful cancer immunotherapy is to induce an effective anti-cancer immunity that will overcome the acquired cancer-specific immune tolerance. In the experiments described herein, it was found that DCs from MM patients suppressed rather than induced a cancer cell-specific immune response. CD4⁺CD25^{high} T cells from MM patients suppressed the proliferation of activated peripheral blood lymphocytes. Further analysis illustrated that MM cell lysates or MM-specific idiotype (Id) immunoglobulins (Igs) specifically induced expansion of peripheral CD4⁺CD25^{high}FoxP3^{high} T regulatory (Treg) cells in vitro. Supraphysiological expression of calnexin using lentiviral vectors in DCs of MM patients overcame the immune suppression and enhanced MM-specific CD4 and CD8 T cell responses. However, over-expression of calnexin did not affect the peripheral expansion of Treg cells stimulated by MM antigens. Thus, the immune suppression effect of Treg cells in cancer patients may be overcome by improving antigen processing in DCs, which in turn may lower the activation threshold of the immune effector cells. This concept of modulating anti-cancer immunity by genetically engineering cancer patients' DCs may improve immunotherapeutic regimens in cancer treatment.

In the studies described herein, CD4⁺CD25⁺Treg and DC functions in MM patients were analyzed and it was demonstrated that CD4⁺CD25⁺Treg had immunosuppressive capacity and that both MM cell lysates and MM-specific Id-Ig loaded DCs triggered a suppressive anti-myeloma immune response exemplified by the expansion of peripheral Treg cells. These tolerizing DC functions, however, could be overcome by lentiviral mediated expression of the calnexin gene in patient's DCs which, in turn, lead to an anti-MM response by the effector T cells. This is the first study to report that engineered DCs from MM patients can overcome peripheral Treg cell-induced immune tolerance.

### Results

### DC Phenotype Analysis in Healthy Donors and MM Patients

For immune analysis, MM patients' monocyte-derived DCs were pulsed with MM cell lysates and cocultured with autologous non-adherent PBMCs as illustrated in FIG. 1A. The MM cells were highly enriched (96-98%) as shown by using antibody-conjugated magnetic beads specific to MM phenotype: CD38⁺CD138⁺CD56⁺CD45⁻ (FIG. 1B). To investigate whether DCs from MM patients were immunologically different, the phenotypes of mature DCs from five healthy donors (HD) and five MM patients (MM) were compared. FIG. 18 illustrates results of CD11c⁺ DC phenotypes from HD and MM patients. Although minor variations existed, there was no significant difference in the surface phenotype between these two groups.

Expansion of CD4⁺CD25^{high}FoxP3^{high} Treg cells by MM cell lysate-pulsed DCs

DCs of MM patients were pulsed with lysates derived from autologous MM cells (DC/MM), autologous normal PBMCs (DC/PBMC) or an allogeneic EBV-transformed B cell line (DC/BCL). Antigen internalization was confirmed by exposure of immature DCs to BCL and double-staining for CD11c and Ig light chains. The kappa or lamda antigens of BCL were efficiently internalized by DCs (4-13.1%) as detected by flow cytometry. Immature DCs were transduced with LV-LacZ expressing a highly immunogenic bacterial β-galactosidase protein for 16-24 h and induced into maturation (DC-LV-LacZ). Similarly treated DCs were exposed to MM cell lysates (DC-LV-LacZ/MM). The DCs were co-cultured with non-adherent autologous PBMCs at a ratio of 1:20 for 14 days and TNF-α- and IFN-γ-producing CD4 and CD8 T cells were detected by intracellular cytokine staining (ICCS) after stimulation with PMA and ionomycin. It was noted that the MM cell lysates (DC/MM) were not immunogenic as the BCL lysates (DC/BCL), and furthermore, T cell activation by DC/LV-LacZ cells was significantly suppressed after exposure to MM cell lysates (FIG. 2A), DC/LV-LacZ versus DC-LV-LacZ/MM).

It is known that during cancer progression, patients may develop strong T regulatory (Treg) activities. Treg-related CD4⁺CD25^{high} lymphocytes in the peripheral blood of MM patients and HD were examined. MM patients showed elevated levels of CD4⁺CD25^{high} lymphocytes (mean 11.51%±1.716%) in the peripheral blood than did HD (mean 6.57%±1.082%; p=0.03). To further examine Treg cells, the expression of FoxP3, an important transcription factor associated with Treg cells, were analyzed. FIG. 2B illustrates a significant increase in gated CD4⁺ T cells with CD25^{high} FoxP3^{high} phenotype when the cells encountered MM cell lysates (DC/MM, mean fluorescence index 8.09 vs. 2.64, 2.83 and 2.95 of DC alone, DC/PBMC and DC/BCL, respectively). Thus, these results suggest that the MM cells selectively induced CD4⁺CD25^{high}FoxP3^{high} Treg cell expansion.

Isolation and Expression of MM-Specific Idiotype Immunoglobulin (MM Id-Ig) Gene

The activation of Treg cells by tumor cell lysate-pulsed DCs may be induced by multiple cancer-related antigens. To see if this effect can be induced by MM Id-Ig antigens, MM specific Id-Ig genes were cloned for further investigation. The MM cells of a kappa-chain specific patient (MM3) were FACS-sorted (CD38⁺CD138⁺CD56⁺CD45⁻, right panel, FIG. 3A), and the RNA was harvested for cDNA synthesis. Specific 5′ V-region and common 3′ C-region primers were used to amplify the MM kappa gene (FIG. 3A). After PCR amplification, one of the six primer pairs (FIG. 3B) revealed a discordant pattern between MM+ (purified MM cells) and MM− cDNAs (MM-minus BM cells). cDNA analysis revealed a consensus CDR3 sequence as underlined in FIG. 3C, resulting from a clonal plasma cells. To confirm this, an oligo-primer specific for the CDR3 sequence of the MM3 patient was used to amplify cDNAs o different MM cells (FIG. 4A). In separate studies, it has been noted that MM patients' BM stromal cells continue to express high level of MM-specific surface markers. Therefore, both BM cells and BM stromal cells were used for this analysis. A positive band was amplified from the corresponding MM3 patient's BM cDNA (MM3 BM cells, L1, FIG. 4B) as well as the corresponding BM stromal cell cDNA (MM3 stromal cells, L2), but not from a different MM patient's (MM4) BM stromal cell cDNA (MM4 stromal cells, L3).

To express myeloma-specific Id-Ig, the MM kappa cDNA was cloned into a lentiviral vector (pTYF-EF) under the control of a strong EF1α promoter. The cDNA was fused with an N-terminal Flag tag (pTYF-EF-k-flag), and the expression confirmed by Western analysis using an anti-Flag antibody (FIG. 4C) with an internal expression control of α-tubulin). Efficient transduction of DCs with lentiviral vectors was demonstrated; up to 40% of DCs were transduced with a reporter LV-eGFP at a multiplicity of infection of 10.

### MM Id-Ig Displays Low Immunogenicity but Induces a Specific Treg Cell Response

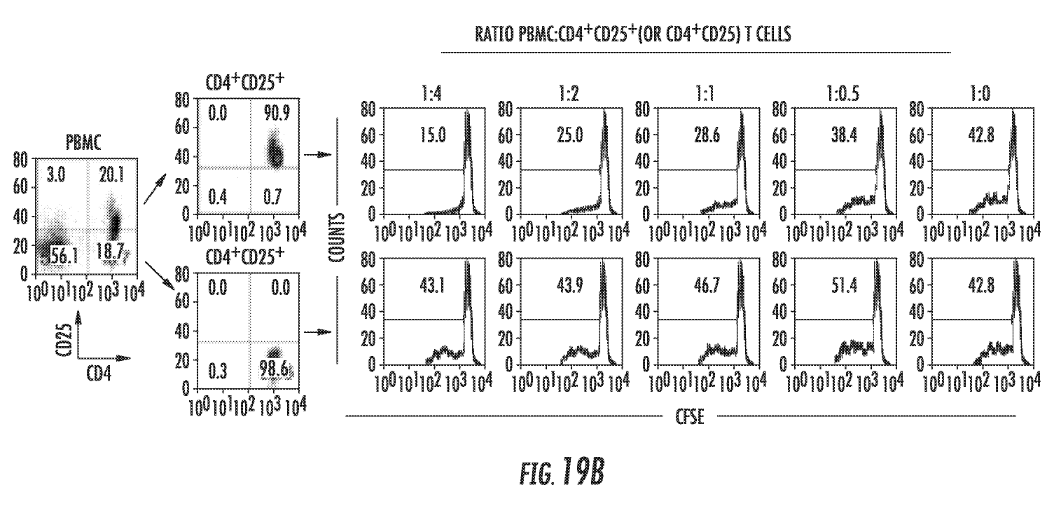

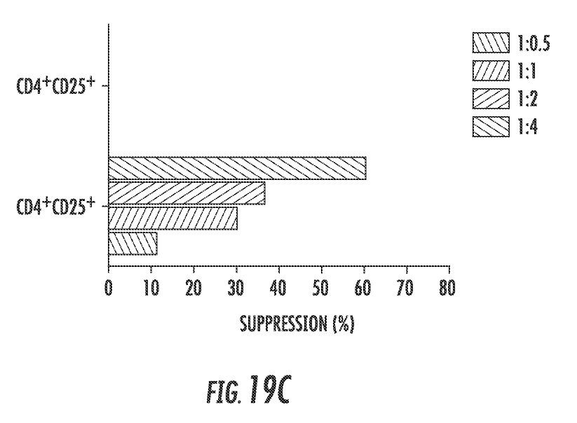

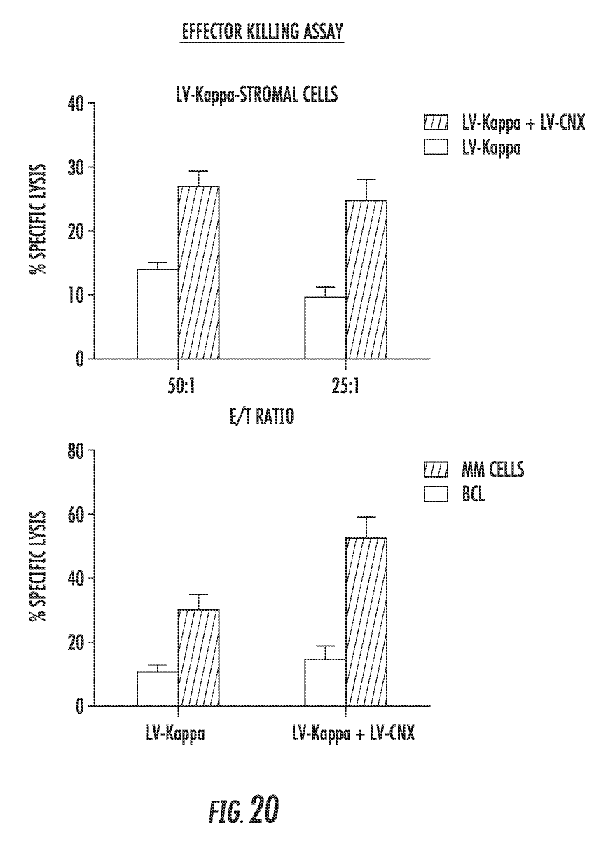

To assess the immunogenicity of the MM Id-Ig, autologous immature DCs were transduced with the MM Id-Ig LV-Kappa vector (DC-LV-Kappa) or a control LV-LacZ vector (DC-LV-LacZ). As positive control, immature DCs were pulsed with memory antigen tetanus toxoid (TT) for 4 h before maturation (DC/TT). The DCs were co-cultured with autologous T cells for 14 days and immune effector function was examined. Background response to the non-transduced DCs was subtracted. For HD there was no difference between DC-LV-Kappa and DC-LV-lacZ in the CD4 and CD8 T cell response (FIG. 19A). In contrast, DC-LV-Kappa induced less CD4 and CD8 T cell response than did DC-LV-LacZ as illustrated by intracellular analysis of TNF-α and IFN-γ (FIG. 19A). Further analysis of CD4⁺CD25⁺ and FoxP3 Treg cells showed that DC-LV-Kappa, but not DC-LV-LacZ or DC/TT, markedly up-regulated FoxP3 expression in CD4⁺CD25^{high} T cells in the corresponding MM patients (MFI of 16.85 for DC-LV-Kappa, vs. 8.23 and 8.21 for DC-LV-LacZ and DC/TT, respectively, p<0.001). In contrast, no significant difference was found for HD (FIGS. 5B and 5C).

It has been reported that CD4⁺CD25⁺ Treg cells of MM patients are dysfunctional. CD4⁺CD25⁺ versus CD4⁺CD25⁻ T cells of MM patients and HD were evaluated in a PBMC proliferation assay. Autologous CD4⁺CD25⁺ or CD4⁺CD25⁻ T cells were co-cultured with CFSE-labeled PBMCs at different ratios and activated with PHA. The intensity of CFSE in the culture decreases with increased cell proliferation. The results showed that CD4⁺CD25⁺ T cells from MM patients suppressed PBMC proliferation in a dose-dependent manner (FIG. 19B and 19C), similar to that of HD. The control CD4⁺CD25⁻ T cells, in contrast, did not show such an effect.

### LV-CNX-Transduced DCs Enhance the MM-Specific CD4 and CD8 T Cell Response

Both MM cell lysates and the specific Id-Ig antigens failed to induce an immune effector response but instead, promoted a strong Treg cell response. To overcome this MM-specific immune suppression, the antigen presentation functions of MM patients' DCs were modified. Calnexin is a chaperone in the ER critical to the processing of glycoproteins and has been shown to promote antigen presentation in immune cells (Bouvier, M. Mol Immunol 39: 697-706, 2003; Williams, D. B. (2006). J Cell Sci 119: 615-623, 2006). The effect of calnexin on MM patients' DCs and T cells was examined by LV-mediated overexpression of calnexin in patients' DCs. The human calnexin cDNA was cloned into lentiviral vector (LV-CNX) behind a strong EF1α promoter. Western analysis detected high expression of calnexin in CEM-NKR cells (a calnexin-defective cell line) after LV-CNX transduction (FIG. 6A). Up-regulation of CNX expression was also confirmed in DCs when transduced with LV-CNX.

To assess the effect of calnexin, immature DCs from MM patients were transduced with LV-CNX (DC-LV-CNX/MM), and control DCs were transduced with LV-LacZ (DC-LV-LacZ/MM), and then pulsed with MM lysates. After maturation, the DCs were cocultured with autologous PBMCs at a ratio of 1:20 for 14 days. The IFN-γ and TNF-α response of the T cells was analyzed upon re-stimulation with PMA and ionomycin. The MM lysate-pulsed DCs again illustrated immune suppression of both CD4 and CD8 T cells (DC-LV-LacZ/MM vs. DC-LV-LacZ, FIG. 6B). Importantly, LV-CNX effectively reversed this trend (FIG. 6B, DC-LV-CNX/MM vs. DC/MM). However, analysis of the LV-CNX effect on Treg cells revealed no reduction in the number of CD4⁺CD25^{high}FoxP3^{high} T cells in coculture with DC-LV-CNX/MM.

### LV-CNX Enhances the MM Id-Ig-Specific DC Immunity

The above study demonstrates that MM patients' DCs, when transduced with LV-CNX can effectively up-regulate MM-specific CD4 and CD8 T cell responses. An enhanced immune activation effect of CNX was observed in a separated study (FIG. 21). Immature DCs were transduced with lentiviral vectors encoding HPV E6E7 (LV-E6/7), a codon-optimized version of E6E7 (LV-Opt E6/7), CNX alone (LV-CNX), or optE6/7 plus CNX (LV-OptE6/7+LV-CNX), and after maturation, co-cultured with autologous non-adherent PBMCs for 14 days. The resulting T cells were re-stimulated with the same antigen-treated DCs. CD4 and CD8 T cell secreting TNF-α and IFN-γ were analyzed by ICCS and flow cytometry (*, p<0.05 and **, p<0.001 and ***, p<0.0001).

T cells were rapidly expanded within three days when LV-CNX-DCs were included in the co-culture (FIG. 7A, clusters of expanded cells). This was verified by CFSE proliferation analysis. The T cells were prestained with CFSE before DC coculture. After three days, the CFSE intensity of the LV-CNX DC coculture group markedly decreased as compared with those of the other three groups, indicating an increased T cell proliferation induced by LV-CNX (FIG. 7A, right panel).

To investigate whether CNX could overcome the tolerance effect of MM-specific antigens, the MM Id-Ig gene (LV-Kappa) was transduced into autologous immature DCs; also included were LV-CNX (DC-LV-Kappa+LV-CNX), and LV-LacZ alone as control (DC-LV-lacZ). The mature DCs were co-cultured with autologous non-adherent PBMCs, and antigen-specific T cell response was examined by ICCS for IFN-γ and TNF-α. The result showed an enhanced response of CD4 and CD8 effector T cells when DCs were co-transduced with LV-CNX (FIG. 7B). The number of CD4⁺CD25^{high}FoxP3^{high} Treg cells between the DC-LV-Kappa+LV-CNX and the DC-LV-Kappa alone, however, was not significantly changed.

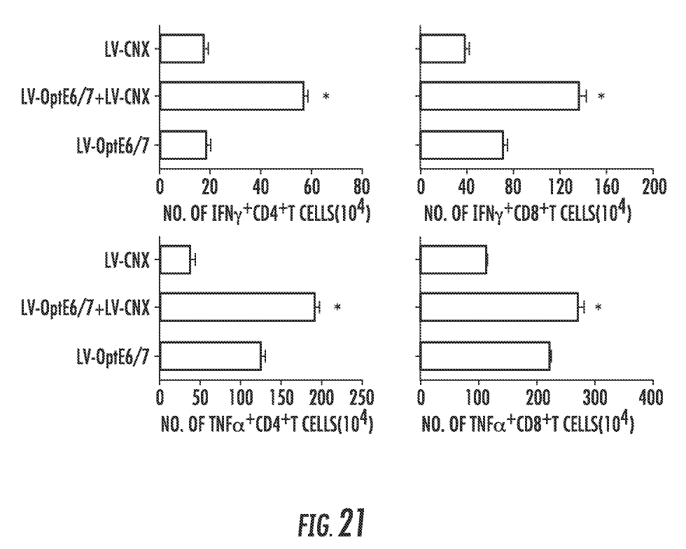

To directly correlate the immune effector function to anti-cancer immunity, the cytotoxic activity of the MM-specific T cells was examined using a non-radioactive target cell killing assay. The T cells were re-stimulated with the corresponding antigen-treated DCs for 5 days and harvested as effector cells. Autologous stromal cells transduced with LV-Kappa (Stromal cells-LV-Kappa) or LV-LacZ (Stromal cells-LV-LacZ) were used as target cells. T cells derived from the DC-LV-Kappa+LV-CNX co-culture killed target Stromal cells-LV-Kappa with increased activity and specificity, as compared with T cells from DC-LV-Kappa at E/T ratio of 50:1 or 25:1 (P<0.03, FIG. 20).

To see if this cytotoxic activity could target primary MM cells, autologous MM cells were isolated from BM (MM cells), and autologous BCL cells were used as control cells. T cells from the coculture of DC-LV-Kappa+LV-CNX killed the MM target cells with increased activity (>40% of specific cell lysis at an E/T ratio of 30:1) as compared with the T cells from the DC-LV-Kappa co-culture (<20%, P<0.02, FIG. 20, bottom graph). Together, these results illustrate that supraphysiological expression of CNX in DCs enhances cytokine production and cytotoxic function of co-cultured immune cells with specificity toward the primary MM cells.

The results described herein indicate that MM cell lysates, or more specifically, the MM-specific Id-Igs induce a Treg cell response instead of an anti-MM response. MM patients' DCs pulsed with MM cell lysates or expressing MM-specific Id-Ig effectively up-regulate CD4⁺CD25^{high}FoxP3⁺Treg cells, which is combined with reduced immune effector functions. This is the first report that clearly demonstrates that MM-specific antigens can selectively expand peripheral Treg cells.

The experiments described herein have demonstrated that the MM immune tolerance can be overcome by modifying DCs to express calnexin, an accessory protein that enhances antigen processing and promotes DC and T cell interactions. MM patients' DCs presenting cancer antigens do not induce an effective T helper or CTL response against cancer cells. LV-CNX-modified MM DCs, in contrast, effectively boosted cytokine production in both CD4 and CD8 T cells coupled with increased cancer cell killing activity. These findings indicate that the tolerogenic DCs in cancer patients may be engineered into reactive DCs to promote an anti-cancer immunity with potential clinical benefit.

In summary, cancer patients including MM patients gradually develop tolerance to their cancer cells with increased Treg activities. This tilted balance of cancer immunity may be altered by properly engineering DCs using LV-CNX. Combining this approach of modified DC vaccine with other ways to modulate the number and/or the functions of Treg may become an effective anti-cancer immunotherapy approach. The results of the experiments described herein support the application of CNX-based immunotherapy in multiple myeloma and other malignancies.

### Materials and Methods

Patients and donors. Bone marrow and peripheral blood were obtained from patients with newly diagnosed or relapsed/refractory multiple myeloma who signed informed consent approved by the Institutional Review Board (IRB) at the University of Florida. Peripheral blood of anonymous healthy donors was obtained from LifeSouth Blood Center, Gainesville, Fla.

Generation of monocyte-derived DCs and bone marrow-derived stromal cells. PBMCs from healthy donors or patients with MM were isolated from buffy coats by gradient density centrifugation in Ficoll-Hypaque (Sigma-Aldrich, St. Louis, Mo.) as previously described (Chen, X., He, J., and Chang, L.-J., Retrovirology 1: 37, 2004). DCs were prepared according to the method of Thurner et al. (Thurneret al., J Immunol Methods 223: 1-15, 1999) with the following modifications: on Day 0, the PBMCs were incubated at 37° C. for 2 h and the adherent monocytic cells were cultured in AIM-V medium. On day 1, one half of the AIM-V medium was supplemented with 50 ng/ml of recombinant human GM-CSF and 25 ng/ml of IL-4 (Biosource International Inc. Camarillo, Calif., USA). On day 3, fresh AIM-V medium containing 100 ng/ml of GM-CSF and 50 ng/ml of IL-4 was added to the culture. On day 5, the non-adherent cells were harvested by gentle pipetting. The purity of immature DCs was routinely examined using fluorochrome-conjugated anti-CD11c Ab (BD Pharmigen, San Diego, Calif.). Immature DCs were induced into maturation with TNF-α (20 U/ml, Biosource International Inc.) and lipopolysaccharide (LPS, 1 μg/ml, Sigma-Aldrich). The phenotype of the mature DCs was verified with fluorochrome-conjugated antibodies against different DC maturation markers including CD1a, CD83, CD80, CD86, CD40 (BD Pharmigen), HLA-I (HLA-ABC) and HLA-DR (Caltag). These DCs are functional in stimulating an antigen-specific T cell response.

BM-derived stromal cells were generated by plating BM cells in α-MEM supplemented with penicillin and streptomycin and 20% fetal bovine serum (FBS), and the attached cells were propagated as stromal cell culture.

Isolation of MM cells and preparation and pulsing of cell lysates. MM cells were enriched from bone marrow mononuclear cells by negative selection with magnetic beads according to the manufacturer's instructions (Stem Cell Technologies, Vancouver, BC) or enriched by FACS with the following mixture of antibodies: FITC-labeled anti-human CD38, PE-labeled anti-human CD138, PE-Cy7 labeled anti-CD56 and APC-labeled anti-CD45 mAb (BD Pharmigen, San Diego, Calif.). These cells and Epstein-Barr virus (EBV) transformed B cell line from different donors were lysed by five rounds of freeze-and-thaw between liquid nitrogen and a 37° C. water bath. Cell debris was discarded by centrifugation (20,800 g, 2 min) and the supernatants were stored frozen at −80° C. until use. The cell lysates were used to pulse immature DCs at ratio of 1:1 (cell number) for 4 h. Tetanus toxoid (TT, inactivated tetanus toxin) was used to pulse the DCs at a concentration 50 U/ml for 4 h. Subsequently, DCs were matured with LPS (1 μg/ml) and TNF-α (20 U/ml) for 24 h.

Isolation and expression of MM-specific idiotype immunoglobulin (Id-Ig) gene. The V_{H} and V_{L} genes of FACS-sorted MM cells were PCR amplified using primers specific for H chain and L chain, followed by cloning and sequencing. The reverse primers of H chain and L chain were complementary to the constant (C) region. The forward primers for H chain and L chain were complementary to the V region of different subfamilies. The sequences of these primers are listed in Table 1.

Lentiviral vector preparation and transduction of DCs. LVs were constructed as described previously (Chang, et al., Gene Therapy 6: 715-728, 1999; Chang, L.-J., and Zaiss, A.-K. Self inactivating lentiviral vectors in combination with a sensitive Cre/loxP reporter system. In Methods in Molecular Medicine (J. Walker, Ed.), pp. 367-382. Humana Press Inc., 2001; Zaiss et al., Journal of Virology 76: 7209-7219, 2002). The self-inactivating pTYF vectors expressing calnexin, MM kappa chain, a Kappa-Flag fusion, nLacZ and eGFP genes were under the EF1α promoter control. The day 5 immature DCs, plated at 5×10⁵ per well in a 24-well plate containing 200 μl of medium supplemented with GM-CSF (50 ng/ml) and IL-4 (25 ng/ml), were transduced with concentrated LVs at a multiplicity of infection (MOI) of 40. The infected cells were incubated at 37° C. for 2 h with gentle shaking every 30 min, followed by adding 1 ml of medium and incubated for an additional 12 h. DC maturation was induced by adding LPS (1 μg/ml) and TNFα (20 μ/ml) and incubated for 24 h.

FACS sort of CD4⁺CD25⁺ lymphocyte subsets. CD4⁺CD25⁺ peripheral blood lymphocytes of healthy donors and MM patients were isolated with a FACSAria high-speed cell sorter (BD Bioscience). Briefly, PBMC were incubated with PE-anti-human CD25 Ab (BD Pharmigen) and APC-anti-human CD4 Ab (Caltag) for 30 min on ice in the dark. The cells were washed three times before sorting. Lymphocytes were gated based on forward and side scatter for further analysis of CD4 and CD25 expression. CD4⁺CD25⁺ and CD4⁺CD25⁻ T cell populations were sorted according to fluorescence of PE (CD25) and APC (CD4). The mean purity of the sorted CD4⁺CD25⁺ and CD4⁺CD25⁻ cells was in the range of 98%.

CFSE labeling-based lymphocyte proliferation assay. PBMC were suspended in PBS containing 0.1% BSA at 2×10⁶/ml and incubated with 5-(and -6)-carboxyfluorescein diacetate succinimidyl ester (CFSE, Molecular Probes, Eugene, Oreg.) at a final concentration of 1 mM for 7 min at 37° C. Cells were washed and resuspended in culture medium for 15 min to stabilize the CFSE staining The CFSE labeled PBMC (responders) were cultured in a 96-well U-shape plate at 4×10⁴ cells/well with phytohemoagglutinin (PHA-P, 1 mg/ml, Sigma-Aldrich, St. Louis, Mo.) in the presence of varying amounts of CD4⁺CD25⁺ T cells (Treg population) and CD4⁺CD25⁻ T cells (control). After 3 days, cells were harvested and CFSE intensity of gated lymphocytes was analyzed by flow cytometry. The suppression effect of Treg was expressed as the relative decrease of CFSE^{low} cells [100×(1−% CFSE^{low} PBMC in coculture/% total CFSE^{low} PBMC)]. CFSE autofluorescence of unlabeled CD4⁺CD25⁺ and CD4⁺CD25⁻ T cells was subtracted using CellQuest software to exclude background interference with CFSE^{low} cells.

DC and non-adherent PBMC coculture. Non-adherent PBMC were cocultured with autologous mature DCs at a ratio of 20:1 in serum-free AIM-V medium for three days. On day 3, IL-7 (10 ng/ml) and IL-2 (12.5 U/ml) were added and fresh medium replenished every other day for 14 days. On day 13, the T cells were collected for Treg cell analysis using fluorochrome-conjugated Abs against CD4, CD25 and FoxP3. On day 14, the T cells were re-stimulated with the same antigen treated mature DCs. For myeloma cell lysate-pulsed DCs, the T cells were re-stimulated with phorbol myristate acetate (PMA, 10 ng/ml or 0.0162 μM) and ionomycin (1 μg/ml, Sigma-Aldrich) for 4 h, with Brefeldin A (1.5 μg/ml) added during the last 2.5 h of culture. Then, the cells were fixed, permeablized, and stained with FITC-labeled anti-IFNγ-, PE-labeled anti-CD8, PE-Cy7-labeled anti-CD4 and APC-labeled anti-TNF-α mAbs (BD Pharmigen). The cells were analyzed using a FACSCalibur flow cytometer (BD Biosciences).

Immune cell cytotoxicity assay. The immune cell cytotoxicity assay was based on a non-radioactive Fluorometric Analysis of T-lymphocyte Antigen-specific Lysis (FATAL assay) as described by Sheehy et al. (J Immunol Methods 249: 99-110, 2001), with modifications (Wang et al., Vaccine 24: 3477-3489, 2006). On day 14 after DC:T cell coculture, the T cells were re-stimulated and 5 days later, harvested as effector cells. The target cells included stromal cells infected with LV-kappa or LV-lacZ, autologous MM cells or Epstein Barr virus (EBV)-transformed B cell line (BLCL). The target cells were labeled with PKH-26 (Sigma-Aldrich) and CFSE (Molecular Probes). The double-labeled target cells were dispensed in duplicate at 1×10⁴ cells/well into 96-well U-bottom plates (BD Biosciences). Effector cells were added at various effector:target (E:T) ratios. After 5 h incubation, the cells were harvested and fixed in 1% paraformaldehyde in PBS and analyzed using a FACSCalibur flow cytometer and the CellQuest program (BD). PKH-26 positive cells were gated and the same cell numbers were acquired for each sample. The percentage of target cell lysis was determined by the disappearance of the antigen specific targets from the CFSE^{high} population compared to the control targets in the CFSE^{high} population.

Western analysis. Cell extracts were prepared in lysis buffer of Cell Signaling Technology, Inc. (Danvers, Mass.) containing proteinase inhibitors (Sigma-Aldrich). The protein samples were separated on sodium dodecyl sulfate 4-12% gradient polyacrylamide gels, electro-blotted to polyvinylidene difluoride membranes (PerkinElmer, Boston, Mass.), and exposed to antibodies against Flag or calnexin (Santa Cruz Biotechnology Inc. Santa Cruz, Calif.). The signals were detected using a horseradish peroxidase kit with enhanced chemiluminescence (Amersham Biosciences, Piscataway, N.J.).

Statistics. Data were analyzed using GraphPad Prism 4 analysis software (GraphPad Software, Inc. San Diego, Calif.) and student's t-test. A 2-sided P value of less than 0.05 was considered statistically significant.

### Other Embodiments

Any improvement may be made in part or all of the compositions and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

1. An antigen presenting cell into which has been introduced at least a first nucleotide sequence that encodes calnexin, wherein expression of calnexin in the antigen presenting cell increases the antigen presenting cell's ability to activate a T cell response.

2. The antigen presenting cell of claim 1, wherein the antigen presenting cell is a dendritic cell.

3. The antigen presenting cell of claim 1, wherein at least a second nucleotide sequence from a lentivirus has been introduced into the antigen presenting cell.

4. The antigen presenting cell of claim 2, wherein the at least first and second nucleotide sequences are comprised within a lentiviral vector.

5. The antigen presenting cell of claim 1, wherein calnexin is expressed at supraphysiological levels.

6. The antigen presenting cell of claim 1, wherein the T cell response is a cancer cell-specific T cell response or a viral-specific T cell response.

7. The antigen presenting cell of claim 6, wherein the T cell response is a cancer cell-specific T cell response and the cancer is multiple myeloma.

8. A method of increasing a cancer cell-specific immune activating activity of an antigen presenting cell, the method comprising the steps of: providing an antigen presenting cell; and introducing into the antigen presenting cell at least a first nucleotide sequence that encodes calnexin, wherein expression of calnexin in the antigen presenting cell increases the antigen presenting cell's ability to activate a T cell response.

9. The method of claim 8 wherein calnexin is expressed at supraphysiological levels in the antigen presenting cell.

10. The method of claim 9, wherein the antigen presenting cell is a dendritic cell.

11. The method of claim 8, wherein the cancer cell is a multiple myeloma cell.

12. The method of claim 8, wherein the at least first nucleotide sequence is comprised within a lentiviral vector.

13. The method of claim 8, wherein the T cell response is a cancer cell-specific T cell response.

14. The method of claim 8, wherein the T cell response is a virus-specific response.

15. The method of claim 8, wherein the at least first nucleotide sequence is introduced into the dendritic cell by a method selected from the group consisting of: infection with a viral vector and transfection with a non-viral vector.


SEQUENCE LISTING (13 sequences)

SEQ ID NO: 1 - DNA, 41 bp, ARTIFICIAL
  OLIGONUCLEOTIDE
ctcgcaactgcctgcagggacatccagatgacccagtctcc

SEQ ID NO: 2 - DNA, 41 bp, ARTIFICIAL
  OLIGONUCLEOTIDE
ctcgcaactgcctgcagggatgttgtgatgactcagtctcc

SEQ ID NO: 3 - DNA, 41 bp, ARTIFICIAL
  OLIGONUCLEOTIDE
ctcgcaactgcctgcagggaaattgtgttgacgcagtctcc

SEQ ID NO: 4 - DNA, 41 bp, ARTIFICIAL
  OLIGONUCLEOTIDE
ctcgcaactgcctgcagggacatcgtgatgacccagtctcc

SEQ ID NO: 5 - DNA, 41 bp, ARTIFICIAL
  OLIGONUCLEOTIDE
ctcgcaactgcctgcagggaaacgacactcacgcagtctcc

SEQ ID NO: 6 - DNA, 41 bp, ARTIFICIAL
  OLIGONUCLEOTIDE
ctcgcaactgcctgcagggaaattgtgctgactcagtctcc

SEQ ID NO: 7 - DNA, 70 bp, ARTIFICIAL
  OLIGONUCLEOTIDE
tcatcctcgacttggccgcctcggccctaacactctcccctgttgaagctctttgtgacgggcgatctca

SEQ ID NO: 8 - DNA, 26 bp, ARTIFICIAL
  OLIGONUCLEOTIDE
aaggatccaccatgctcgcaactgcc

SEQ ID NO: 9 - DNA, 33 bp, ARTIFICIAL
  OLIGONUCLEOTIDE
aaactagtcactaacactctcccctgttgaagc

SEQ ID NO: 10 - DNA, 20 bp, ARTIFICIAL
  OLIGONUCLEOTIDE
agtatgatgttctcccatac

SEQ ID NO: 11 - DNA, 51 bp, ARTIFICIAL
  OLIGONUCLEOTIDE
aaactagtctacttgtcgtcatcgtctttgtagtcacactctcccctgttg

SEQ ID NO: 12 - DNA, 705 bp, HOMO SAPIENS
atgctgcagacgcgttacgtatcggatccagaattcgtgattctcgcaactgcctgcagggaaattgtgctgactcagtctccatcctccctgtctgcgtctgtaggagacagagtcaccatcacttgccaggccagtcaggacataagcaactttttaatttggtatcaacaaaaaccagggaaagcccctaaactcctcatctacgatgcaaccgatttggaagcgggggtcccatcaaggttcactggtagcgggtccgggacacactttagtctcaccatcaccagcctgcagcgtgaagatgttgcaacatatttctgtcaacagtatgatgttctcccatacacgtttggccaggggaccaaactgggggtcaaacgaactgtggctgcaccatctgtcttcattttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagatcgcccgtcacaaagagcttcaacaggggagagtgttag

SEQ ID NO: 13 - protein, 234 aa, HOMO SAPIENS
MLQTRYVSDPEFVILATACREIVLTQSPSSLSASVGDRVTITCQASQDISNFLIWYQQKPGKAPKLLIYDATDLEAGVPSRFTGSGSGTHFSLTITSLQREDVATYFCQQYDVLPYTFGQGTKLGVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC